(19) United States
(12) Patent Application Publication
(10) Pub. No.: US 2010/0028402 A1
(43) Pub. Date: Feb. 4, 2010

(54) NANOPARTICLE-BASED ANTICOAGULANT

(76) Inventors: Marina Dobrovolskaia, Frederick, MD (US); Scott McNeil, Frederick, MD (US); Barry W. Neun, Hampstead, MD (US)

Correspondence Address: KLARQUIST SPARKMAN, LLP, 121 S.W. SALMON STREET, SUITE #1600, PORTLAND, OR, 97204-2988, US

(21) Appl. No.: 12/446,967

(22) PCT Filed: Oct. 25, 2006
(86) PCT No.: PCT/US06/41838

§ 371 Date: Apr. 23, 2009

(51) Int. Cl. A61K 9/14; A61K 9/00; A61K 31/785; A61P 7/02; A61K 31/19

(52) U.S. Cl. 424/423; 424/422; 424/501; 424/78.08; 424/78.17; 514/574

(57) ABSTRACT

A method for preventing or treating a blood clotting disorder is disclosed. The method includes administering a therapeutic effective amount of at least one nanoparticle-based anticoagulant to a subject afflicted with blood clotting disorder or potentially afflicted with a blood clotting disorder, wherein the at least one nanoparticle-based anticoagulant is a substituted fullerene, polyamidoamine (PAMAM) dendrimer or combination thereof.

### FIELD

The present disclosure relates to the use of nanoparticles and in particular, to substituted fullerenes and polyamidoamine dendrimers and their use to inhibit blood clotting disorders such as thrombosis.

### BACKGROUND

Nanotechnology has been suggested to be one of the critical research endeavors of the early 21st century, as scientists reveal the unique properties of atomic and molecular assemblages built at the nanometer scale. Nanotechnology is often defined as research and technology development at the atomic, molecular, or macromolecular scale, leading to the controlled creation and use of structures, devices, and systems with a length scale of 1 to 100 nanometers (nm). Nanotechnology manifests itself in a wide range of materials (such as carbon nanotubes and gold nanoshells) and particles (such as fullerenes and dendrimers).

Buckminsterfullerenes more commonly referred to as fullerenes or “buckyballs” are cage-like molecules formed primarily of sp²-hybridized carbons. Commonly known fullerenes include C₆₀ and C₇₀. Cₙ refers to a fullerene moiety including n number of carbon atoms. Methods of substituting fullerenes with various substituent groups are known in the art. C₆₀ as well as C₆₀-substituted derivatives have been suggested for treating a number of medical disorders. For example, C₆₀ and C₆₀-substituted derivatives have been shown to be capable of reacting with oxygen radicals associated with various oxidative stress diseases.

Dendrimers are nanoparticles that are composed of a central core and branched monomers. Each dendrimer is globular in shape and includes a large number of end groups known as surface or terminal groups. This configuration is the result of the cyclic manner in which the dendrimer is built. The more branches added to the core, the higher generation of dendrimer formed. For example, polyamidoamine dendrimers are based on an ethylenediamine core, and branched units are constructed from methyl acrylate and ethylenediamine (Tomalia, D. A. et al. Polym. J. 17:117-132 (1985). The specific structure of the dendrimer has been suggested to make dendrimers suitable for a variety of biomedical applications including oligonucleotide transfection agents and drug carriers.

The ability to manipulate the physical, chemical, and biological properties of nanoparticles has allowed researchers to begin to design and use nanoparticles for various therapeutic and diagnostic purposes. However, therapeutic and diagnostic uses for particular nanoparticles such as substituted fullerenes and dendrimers remain to be fully elucidated.

### SUMMARY

Disclosed herein are methods for preventing or treating a blood clotting disorder. In an example, a method includes administering a therapeutic effective amount of at least one nanoparticle-based anticoagulant to a subject afflicted with blood clotting disorder or potentially afflicted with a blood clotting disorder, wherein the at least one nanoparticle-based anticoagulant is a substituted fullerene, polyamidoamine (PAMAM) dendrimer or combination or mixture thereof. Illustrative nanoparticle-based anticoagulants include C3, a C3 analog, DF1, a DF1 analog, NCL22, a NCL22 analog or a combination or mixture thereof. The blood clotting disorder can be associated with the implantation of a medical device such as a stent or a catheter. Further, the disorder can range from undesired platelet aggregation to more serious disorders such as thrombosis or peripheral arterial occlusion. The nanoparticle-based anticoagulant can be administered via traditional drug delivery routes such as intravenously or the anticoagulant can be administered by the medical device. For example, the medical device can be at least partially coated or impregnated with the nanoparticle-based anticoagulant.

The foregoing and other features and advantages will become more apparent from the following detailed description, which proceeds with reference to the accompanying figures.

### BRIEF DESCRIPTION OF THE DRAWINGS

FIG. 1 is a schematic representation of substituted C₆₀ compounds including DF1, C3, AF1 and AF3.

FIG. 2 is a bar graph showing the effects of the substituted C₆₀ compounds illustrated in FIG. 1 on red blood cell integrity.

FIG. 3 is a bar graph illustrating the effects of the substituted C₆₀ compounds illustrated in FIG. 1 on bone marrow myeloid precursors.

FIG. 4 is a bar graph illustrating the effect of the substituted C₆₀ compounds illustrated in FIG. 1 on platelet aggregation in the presence and absence of collagen.

FIG. 5 is a bar graph illustrating the effect of aspirin on collagen-induced platelet aggregation.

FIG. 6 is a schematic representation of the carboxy-terminated PAMAM dendrimer NCL22.

FIG. 7 is a mass spectrum profile of NCL22.

FIG. 8 is a bar graph illustrating the effect of NCL22 on red blood cell integrity.

FIG. 9 is a bar graph illustrating the effect of NCL22 on cytokine secretion.

FIG. 10 is a bar graph illustrating the effect of NCL22 on macrophage chemotactic activities.

FIG. 11 is a bar graph showing the effect of NCL22 on the phagocytic uptake of Zymosan-A.

FIG. 12 is a bar graph showing the effect of NCL22 on leukocyte proliferation.

FIG. 13 is a bar graph illustrating the effects of NCL22 on growth and differentiation of bone marrow myeloid progenitors.

FIG. 14 is a bar graph showing the effect of NCL22 on platelet aggregation.

FIG. 15 is a bar graph illustrating the effect of NCL22 on coagulation.

FIG. 16A is a digital image of proteins separated on a two dimensional polyacrylamide gel following isolation of the proteins after NCL22 treatment and electrophoresis.

FIGS. 16B and C are digital images of proteins separated on a two dimensional polyacrylamide gel following exposure to acetic acid or no blocking buffers and electrophoresis.

FIG. 17 is a graph illustrating the cytotoxic effects of NCL22 on LDK-PK1 kidney epithelial cells according to a MTT cytotoxicity assay.

FIG. 18 is a graph illustrating the cytotoxic effects of NCL22 on LDK-PK1 kidney epithelial cells according to a LDH cytotoxicity assay.

FIG. 19 is a graph illustrating the cytotoxic effects of NCL22 on HepG2 hepatic carcinoma cells according to a MTT cytotoxicity assay.

FIG. 20 is a graph illustrating the cytotoxic effects of NCL22 on HepG2 hepatic carcinoma cells according to a LDH cytotoxicity assay.

### DETAILED DESCRIPTION

### I. Introduction

The coagulation of blood is a complex process during which blood forms solid clots. It is an important part of hemostasis whereby a damaged blood vessel wall is covered by a fibrin clot to stop hemorrhaging and aid repair of the damaged vessel. Disorders in coagulation can lead to increased hemorrhage and/or thrombosis and embolism. For example, peripheral arterial occlusion (PAO), also known as “leg attack”, is a significant cause of morbidity and amputation in the United States with more than 100,000 cases reported annually. It occurs when arterial blood flow to a distant part of the body, such as the leg, is blocked by a clot. Current treatment for acute PAO includes invasive open vascular surgery or off-label use of thrombolytic drugs. Although there is a strong demand for new anti-coagulant therapies for prevention and treatment of PAO, currently no thrombolytic agents are specifically approved by the FDA for treatment of acute PAO.

Drugs that inhibit platelets from aggregating to form a plug are used to prevent undesired blood clotting as well as to alter the natural course of atherosclerosis (local platelet aggregation is an early step in the atherosclerotic plaque formation).

Disclosed herein are nanoparticle-based anticoagulants that can interfere with the contact activation pathway of coagulation. For example, substituted fullerenes C3 and DF1 are reported to interfere with collagen-induced platelet aggregation. The PAMAM dendrimer NCL22 is disclosed as slowing coagulation time as well as being an effective inhibitor of plasma protein binding. It is believed that such anticoagulants can be administered to prevent and/or treat blood clotting disorders such as PAO or thrombosis. In addition, the disclosed methods can also be employed to prevent and/or treat other blood clotting disorders such as those associated with implantation of a medical device. For example, the implantation device can be coated or impregnated with the nanoparticle-based anticoagulant to prevent and/or treat undesired platelet aggregation or thrombosis at the implantation site. A nanoparticle-based anticoagulant can also be locally administered at the site of the device.

### II. Abbreviations and Terms

### a. Abbreviations

CMH cyanmethemoglobin
CFU-GM colony-forming unit-granulocyte-macrophage
FBS fetal bovine serum
IL interleukin
LPS lipopolysaccharide
mM millimolar
PAMAM polyamidoamine
PBS phosphate buffer saline
PFH plasma free hemoglobin
TBH total blood hemoglobin
TNF tumor necrosis factor

### b. Terms

Unless otherwise explained, all technical and scientific terms used herein have the same meaning as commonly understood by one of ordinary skill in the art to which the disclosed subject matter belongs. Definitions of common terms in chemistry terms may be found in The McGraw-Hill Dictionary of Chemical Terms, 1985, and The Condensed Chemical Dictionary, 1981. As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description. Except as otherwise noted, the methods and techniques of the present invention are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

In order to facilitate review of the various embodiments of this disclosure, the following explanations of specific terms are provided:

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Anticoagulant: A substance that prevents the clotting of blood (coagulation). Anticoagulants are commonly administered to subjects to prevent or treat thrombosis. Generally, anticoagulants are administered to treat or prevent deep vein thrombosis, pulmonary embolism, myocardial infarction, stroke, and mechanical prosthetic heart valves. Various types of anticoagulants with different mechanisms of action are available including anticoagulants that inhibit the effect of vitamin K (such as coumadin) or thrombin directly (such as argatroban, lepirudin, bivalirudin, and ximelagatran) or that activate antithrombin II that in turn blocks thrombin from clotting blood (such as heparin and derivative substances thereof). In an example, an anticoagulant is a nanoparticle-based anticoagulant such as a substituted fullerene or PAMAM dendrimer.

Blood vessel: The vessels through which blood circulates. In general, blood vessels are elastic tubular channels that are lined with endothelium. Blood vessels include the arteries, veins and capillaries. Specific, non-limiting examples of a blood vessel include a vena cave, a thoracic aorta, a saphanous vein, a mammary artery, the brachial artery and a capillary. In another embodiment, a blood vessel includes the smaller arteries and veins. In yet another embodiment, a blood vessel is a capillary of the microvascular circulation.

Blood clotting disorder: A disease resulting in a defect in hemostasis in a subject. In one embodiment, it is the abnormal clotting of blood, such as clotting of blood within an artery or vein. Normal blood hemostasis is a complex process involving as many as 20 different plasma proteins, known as clotting factors. Normally, a complex chemical process occurs using these clotting factors to form a substance called fibrin that stops bleeding. Fibrin formation can also aid in repair of a damaged blood vessel. Disorders in coagulation can lead to increased hemorrhage and/or thrombosis and embolism. The “contact activation pathway” of coagulation is initiated by platelets adhering to and activated by collagen in the endothelium of a blood vessel. In the present disclosure, nanoparticles such as fullerenes can interfere with collagen-induced coagulation.

C3 compound: A substituted fullerene produced by the precise grafting of three malonic acid groups to the C₆₀ fullerene surface.

Catheter: A tube that can be inserted into a body cavity duct or vessel thereby allowing drainage or injection of fluids or access by surgical instruments. Catheters are widely used in medical applications, e.g., for intravenous, arterial, peritoneal, pleural, intrathecal, subdural, urological, synovial, gynecological, percutaneous, gastrointestinal, abscess drains, and subcutaneous applications. Catheters are placed for short-term, intermediate, and long-term usage. Types of catheters include standard intravenous (IV), peripherally inserted central catheters (PICC)/midline, central venous catheters (CVC), angiographic catheters, guide catheters, feeding tubes, endoscopy catheters, Foley catheters, drainage catheters, and needles. Catheter complications include phlebitis, localized infection and thrombosis.

Coat: As used herein “coating”, “coatings”, “coated” and “coat” are forms of the same term defining material and process for making a material where a first substance or substrate surface is at least partially covered or associated with a second substance. Both the first and second substance are not required to be different. Further, when a device is “coated” as used herein, the coating may be effectuated by any chemical or mechanical bond or force, including linking agents. Thus a device composed of a first substance may be “coated” with a second substance via a linking agent that is a third substance. As used herein, the “coating” need not be complete or cover the entire surface of the first substance to be “coated”. The “coating” may be complete as well (e.g., approximately covering the entire first substance). There can be multiple coatings and multiple substances within each coating. The coating may vary in thickness or the coating thickness may be substantially uniform.

Coatings contemplated in accordance with the present disclosure include, but not limited to medicated coatings, drug-eluting coatings, drugs or other compounds, pharmaceutically acceptable carriers and combinations thereof, or any other organic, inorganic or organic/inorganic hybrid materials. In an example, the coating includes nanoparticles on a medical device. For example, in a preferred embodiment for anticoagulation properties, a medical device is coated with nanoparticles that have anticoagulant properties such as C3, DF1, NCL22 or a combination thereof.

DF1 compound: A dendrofullerene produced by attaching a highly water-soluble conjugate to the C₆₀ fullerene core. In pre-clinical testing, DF1 has been demonstrated to be highly soluble, nontoxic, and able to retain a high level of antioxidant activity in both cultured cells and animals.

Dendrimer: A specific class of polymers that include a central core and branched monomeric units. Numerous families of dendrimers have been synthesized with various core molecules and building monomers. For example, polyamidoamine (PAMAM) dendrimers are based on an ethylenediamine core, and branched units constructed from both methyl acrylate and ethylenediamine. The manufacturing process is a series of repetitive steps starting with a central initiator core. Each subsequent growth step represents a new “generation” of polymer with a larger molecular diameter (the diameter increases by about 10), twice the number of reactive surface sites, and approximately double the molecular weight of the preceding generation.

As dendrimers grow in generations, the subsequent increase in exterior branching density begins to impart various structural effects to the polymer shape. Lower generation dendrimers, on the order of 0 through 4 show a flexible, flat shape, while at the higher generations of 5 through 10, the congested branching induces a persistent, spherical conformation. Beginning at generation 4 using an ethylenediamine core, the interior of the dendrimer develops inter void spaces that are accessible to molecules that may be encapsulated for various uses. The half-generations of PAMAM dendrimers, such as NCL22, possess surfaces of carboxylate groups and the full-generations of PAMAM dendrimers possess surfaces of amino groups. NCL22 is a fourth and a half generation (G4.5) PAMAM dendrimer with 64 carboxy end groups on its surface. The Table below shows the calculated properties of amine surface functional PAMAM dendrimers by generation.

| | Molecular | Measured Diameter | |
|---|---|---|---|
| Generation | Weight | (Å) | Surface Groups |
| 0 | 517 | 15 | 4 |
| 1 | 1,430 | 22 | 8 |
| 2 | 3,256 | 29 | 16 |
| 3 | 6,909 | 36 | 32 |
| 4 | 14,215 | 45 | 64 |
| 5 | 28,826 | 54 | 128 |
| 6 | 58,048 | 67 | 256 |
| 7 | 116,493 | 81 | 512 |
| 8 | 233,383 | 97 | 1024 |
| 9 | 467,162 | 114 | 2048 |
| 10 | 934,720 | 135 | 4096 |

Dendron: An addendum of the fullerene which has a branching at the end as a structural unit. Dendrons can be considered to be derived from a core, wherein the core contains two or more reactive sites. Each reactive site of the core can be considered to have been reacted with a molecule including an active site (in this context, a site that reacts with the reactive site of the core) and two or more reactive sites, to define a first generation dendron. First generation dendrons are within the scope of the term “dendron,” as used herein. Higher generation dendrons can be considered to have formed by each reactive site of the first generation dendron having been reacted with the same or another molecule comprising an active site and two or more reactive sites, to define a second generation dendron, with subsequent generations being considered to have been formed by similar additions to the latest generation. Although dendrons can be formed by the techniques described above, dendrons formed by other techniques are within the scope of “dendron” as used herein.

The core of the dendron is bonded to the fullerene by one or more bonds between (a) one or more carbons of the fullerene and (b) one or more atoms of the core. In one example, the core of the dendron is bonded to the fullerene in such a manner as to form a cyclopropanyl ring. In another example, the core of the dendron includes, between the sites of binding to the fullerene and the reactive sites of the core, a spacer that can be a chain of 1 to about 100 atoms, such as about 2 to about 10 carbon atoms. The generations of the dendron can comprise trivalent or polyvalent elements such as, for example, N, C, P, Si, or polyvalent molecular segments such as aryl or heteroaryl. The number of reactive sites for each generation can be about two or about three. The number of generations can be between 1 and about 10, inclusive. More information regarding dendrons suitable for adding to fullerenes can be found in Hirsch et al., U.S. Pat. No. 6,506,928, the disclosure of which is hereby incorporated by reference in its entirety.

Fullerene: Buckminsterfullerenes, also known as fullerenes or, more colloquially, buckyballs, are cage-like molecules consisting essentially of sp²-hybridized carbons and have the general formula (C₂₀₊₂ₘ) (where m is a natural number). Fullerenes are the third form of pure carbon, in addition to diamond and graphite. Typically, fullerenes are arranged in hexagons, pentagons, or both. Most known fullerenes have 12 pentagons and varying numbers of hexagons depending on the size of the molecule. “Cₙ” refers to a fullerene moiety comprising n carbon atoms. Common fullerenes include C₆₀ and C₇₀, although fullerenes comprising up to about 400 carbon atoms are also known. Fullerenes can be produced by any known technique, including, but not limited to, high temperature vaporization of graphite. Fullerenes are available from CSixty Corporation (Houston, Tex.) and MER Corporation (Tucson, Ariz.), among other sources.

A substituted fullerene is a fullerene having at least one substituent group bonded to at least one carbon of the fullerene core. Exemplary substituted fullerenes include carboxyfullerenes, hydroxylated fullerenes, dendritic fullerene, among others. A carboxyfullerene, as used herein, is a fullerene derivative comprising a Cₙ core and one or more substituent groups, wherein at least one substituent group comprises a carboxylic acid moiety or an ester moiety. C3 is an example of a carboxyfullerene. Generally, carboxyfullerenes are water soluble, although whether a specific carboxyfullerene is water soluble is a matter of routine experimentation for the skilled artisan. A dendritic fullerene includes a fullerene core (Cn), and from 1 to 6 dendrons bonded to the fullerene core. DR-1 is an example of a dendritic fullerene.

Medical device/Indwelling device: A device that is introduced temporarily or permanently into a subject for the prophylaxis or therapy of a medical condition. The term medical device may also encompass an “indwelling device.” A medical/indwelling device can include any device that is introduced subcutaneously, percutaneously or surgically to rest within an organ, tissue or lumen of an organ, such as an artery, vein, ventricle, or atrium of the heart. For example, a medical device can include, but is not limited to, a stent, a stent graft, a synthetic vascular graft, a heart valve, a catheter, a vascular prosthetic filter, a pacemaker, a pacemaker lead, a defibrilator, a patent foramen ovale (PFO) septal closure device, a vascular clip, a vascular aneurysm occluder, a hemodialysis graft, a hemodialysis catheter, an atrioventricular shunt, an aortic aneurysm graft device or components, a venous valve, a suture, a vascular anastomosis clip, an indwelling venous or arterial catheter, a vascular sheath and a drug delivery port. The medical device can be made of numerous materials depending on the device. For example, a stent can be made of stainless steel, Nitinol (NiTi), or chromium alloy. Synthetic vascular grafts can be made of a cross-linked PVA hydrogel, polytetrafluoroethylene (PTFE), expanded polytetrafluoroethylene (ePTFE), porous high density polyethylene (HDPE), polyurethane, and polyethylene terephthalate. It is contemplated that the device may be made of nanostructures including nanotubes. It is further contemplated that the device can be coated or impregnated with an anticoagulant substance (such as the disclosed nanoparticle-based anticoagulants).

Nanoparticle: A nanoparticle is a microscopic particle whose size is measured in nanometres (nm). It is defined as a particle that does not have a dimension >200 nm, particularly >100 nm, and more particularly >10 nm. In examples, nanoparticles do not have a dimension less than 9 nm, 8 nm, 7 nm, 6 nm, 5 nm, 4 nm, 3 nm, or 2 nm. For example, the dimension of C3 is 1.57 nm, DF1 5.8 nm and NCL22, 5.97 nm. Nanoparticles are effectively a bridge between bulk materials and atomic or molecular structures. A bulk material should have constant physical properties regardless of its size, but at the nano-scale this is often not the case. Size-dependent properties are observed such as quantum confinement in semiconductor particles, surface plasmon resonance in some metal particles and superparamagnetism in magnetic materials. Semi-solid and soft nanoparticles have been manufactured. A prototype nanoparticle of semi-solid nature is the liposome.

At the small end of the size range, nanoparticles are often referred to as clusters. Metal, dielectric, and semiconductor nanoparticles have been formed, as well as hybrid structures (e.g., core-shell nanoparticles). Nanospheres, nanorods, and nanocups are just a few of the shapes that have been grown. Semiconductor quantum dots and nanocrystals are types of nanoparticles. Such nanoscale particles are used in biomedical applications as drug carriers or imaging agents. Various types of liposome nanoparticles are currently used clinically as delivery systems for anticancer drugs and vaccines.

Nanoparticle characterization is necessary to establish understanding and control of nanoparticle synthesis and applications. Characterization is done by using a variety of different techniques, mainly drawn from materials science. Common techniques are electron microscopy [TEM,SEM], atomic force microscopy [AFM], dynamic light scattering [DLS], x-ray photoelectron spectroscopy [XPS], powder x-ray diffractometry [XRD], and Fourier transform infrared spectroscopy [FTIR].

Nanoparticle-based anticoagulant: An anticoagulant that includes a nanoparticle. In an example, the nanoparticle is a substituted fullerene such as C3, a C3 analog, DF1, or a DF1 analog. In another example, the nanoparticle is a PAMAM dendrimer such as NCL22. In a further example, the nanoparticle-based anticoagulant is a combination or mixture of the substituted fullerenes or at least one substituted fullerene and a PAMAM dendrimer. In an additional example, the nanoparticle-based anticoagulant is a nanoparticle including carboxy-terminated dendritic branches.

NCL22: A polyamidoamine (PAMAM) dendrimer that possesses surfaces of carboxylate groups and full-generation-surfaces of amino groups. NCL22 is a fourth and a half generation (G4.5) PAMAM dendrimer with 64 carboxy end groups on its surface.

Peripheral Arterial Occlusion (PAO): A medical condition (commonly known as “leg attack”) that occurs when arterial blood flow to a distant part of the body, such as the leg, is blocked by a clot. Current treatment for acute PAO includes invasive open vascular surgery or off-label use of thrombolytic drugs. There are currently no thrombolytic agents specifically approved by the FDA for treatment of acute PAO. In the present disclosure, substituted fullerenes (such as C3 or DF1) and PAMAM dendrimers (such as NCL22) are suggested as thrombolytic agents that may be used to treat acute PAO.

Pharmaceutically acceptable dose and/or carrier or adjuvant: Compounds, materials, compositions, and/or dosage forms which are, within the scope of sound medical judgment, suitable for use, in contact with the tissues of human beings or animals without excessive toxicity, irritation, allergic response, or other problem or complication, commensurate with a reasonable benefit/risk ratio. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations usually; contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Platelet Aggregation: The clustering of thrombocytes to facilitate blood coagulation. Platelet aggregation is essential in the clotting of blood at the site of an injury because at the site of injury, the platelets will clump together, swell and stick to the injured area, acting as plug to reduce bleeding. Platelet aggregation assays/tests help to diagnose diseases of platelet dysfunction and distinguish between inherited bleeding problems (such as hemophilia or von Willebrand disease) and acquired bleeding problems (those that occur because of another disorder or as a side-effect of medication). As used herein, “undesired platelet aggregation” may include aggregation that could result in an adverse physiological condition such as thrombosis or blockage of a medical device as determined by platelet aggregation assays, antiphospholipid profile studies, air plethysmography or like procedures.

Subject: A term that includes both human and veterinary subjects. For example, “a subject being treated” is understood to include all animals (e.g., humans, apes, dogs, cats, horses, and cows) that require an increase in the desired biological effect, such as enhanced inhibition of platelet aggregation.

Therapeutically effective amount/dose: An amount/dose of a pharmaceutical preparation that alone, or together with a pharmaceutically acceptable carrier or one or more additional therapeutic agents, induces the desired response. A therapeutic agent, such as a nanoparticle-based anticoagulant, is administered in therapeutically effective amounts. Effective amounts of a therapeutic agent can be determined in many different ways, such as assaying for a reduction in platelet aggregation or improvement of physiological condition of a subject having thrombosis. Effective amounts also can be determined through various in vitro, in vivo or in situ assays. Therapeutic agents can be administered in a single dose, or in several doses, for example daily, during a course of treatment. However, the effective amount can be dependent on the source applied, the subject being treated, the severity and type of the condition being treated, and the manner of administration. In one example, it is an amount sufficient to partially or completely alleviate thrombosis associated with implantation of a medical device within a subject. Treatment can involve only slowing the progression of a blood clotting disorder temporarily, but can also include halting or reversing the progression of a blood clotting disorder permanently. For example, a pharmaceutical preparation can decrease one or more symptoms of the blood clotting disorder (such as thrombosis), for example decrease a symptom by at least 20%, at least 50%, at least 70%, at least 90%, at least 98%, or even at least 100%, as compared to an amount in the absence of the pharmaceutical preparation.

Thrombin inhibitor: A product that is potentially or actually pharmaceutically useful as an inhibitor of thrombin, and includes reference to substance which comprises a pharmaceutically active species and is described, promoted or authorized as a thrombin inhibitor. Such thrombin inhibitors may be selective, that is they are regarded, within the scope of sound pharmacological judgment, as selective towards thrombin in contrast to other proteases; the term “selective thrombin inhibitor” includes reference to substance which comprises a pharmaceutically active species and is described, promoted or authorized as a selective thrombin inhibitor.

Thrombosis: The abnormal clotting of blood within an artery or vein that can reduce or stop the flow of blood within the vessel. For example, thrombosis affecting the coronary arteries can cause a heart attack. Thrombosis affecting the arteries supplying the brain with blood can lead to a stroke.

Treatment: A therapeutic intervention that ameliorates a sign or symptom of a disease or pathological condition, such as a sign, parameter or symptom of thrombosis. Treatment can also induce remission or cure of a condition, such as thrombosis and prevention of the onset of a disease or pathological condition. Treatment of a subject includes treating a subject afflicted with a disease or pathological condition and treating a subject who is at risk for a disease or pathological condition.

Stent: An endovascular support device that is employed to enhance and support existing passages, channels, and conduits such as the lumen of a blood vessel. In an example, a stent is effective to maintain a vessel open, without resulting in significant thrombosis at the affected vessel. In present disclosure, a stent can be coated with or impregnated with a nanoparticle-based anticoagulant to prevent thrombosis from developing with stent implantation.

### III. Nanoparticle-Based Anticoagulants

A. Chemical Structure

Some exemplary nanoparticle-based anticoagulants include substituted fullerenes. In an example, a substituted fullerene comprises a fullerene core (Cₙ) that can have any number of carbon atoms (n), wherein n is an even integer greater than or equal to 60. In an example, the Cₙ has 60 carbon atoms (and may be represented as C₆₀). In another example, the Cₙ has 70 carbon atoms (and may be represented as C₇₀).

Throughout this description, particular examples described herein may be described in terms of a particular acid, amide, ester, or salt conformation, but the skilled artisan will understand an embodiment can change among these and other conformations: depending on the pH and other conditions of manufacture, storage, and use. All such conformations are within the scope of the appended claims. The conformational change between, e.g., an acid and a salt is a routine change, whereas a structural change, such as the decarboxylation of an acid moiety to —H. is not a routine change.

In one example, the substituted fullerene comprises a fullerene core (Cₙ) and m (>CX¹X²) groups bonded to the fullerene core. The notation “>C” indicates the group is bonded to the fullerene core by two single bonds between the carbon atom “C” and the Cₙ. The value of m can be an integer from 1 to 6, inclusive.

X¹ can be selected from —H; —COOH; —CONH₂; —CONHR′; —CONR′₂; —COOR′; —CHO; —(CH₂)_{d}OH; a peptidyl moiety; a heterocyclic moiety; a branched moiety comprising one or more terminal —OH, —NH2, triazole, tetrazole, or sugar groups; or a salt thereof, wherein each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol, and d is an integer from 0 to about 20. In one example, X¹ can be selected from —R, —RCOOH, —RCONH₂, —RCONHR′, —RCONR′₂, —RCOOR′, —RCHO, —R(CH₂)_{d}OH, a peptidyl moiety, or a salt thereof, wherein R is a hydrocarbon moiety having from 1 to about 6 carbon atoms. In another example, X¹ can be selected from —H; —COOH; —CONH₂; —CONHR′; —CONR′₂; —COOR′; —CHO; —(CH₂)_{d}OH; a peptidyl moiety; —R; —RCOOH; —RCONH₂; —RCONHR′; —RCONR′₂; —RCOOR′; —RCHO; —R(CH₂)_{d}OH; a heterocyclic moiety; a branched moiety comprising one or more terminal —OH, —NH₂, triazole, tetrazole, or sugar groups, or a salt thereof.

A heterocyclic moiety is a moiety comprising a ring, wherein the atoms forming the ring are of two or more elements. Common heterocyclic moieties include those comprising: carbon and nitrogen, among others.

A branched moiety is a moiety comprising at least one carbon atom which is bonded to three or four other carbon atoms, wherein the moiety does not comprise a ring. In one example, the branched moiety comprising one or more terminal —OH, —NH₂, triazole, tetrazole, or sugar groups can be selected from —R(CH₂)_{d}C(COH)_{g}(CH₃)_{g-3}, —R(CH₂)_{d}C(CNH₂)_{g}(CH₃)_{g-3}, —R(CH₂)_{d}C(C[tetrazol])_{g}(CH₃)_{g-3}, —R(CH₂)_{d}C(C[triazol])_{g}(CH₃)_{g-3}, R(CH₂)C(C[hexose])_{g}(CH₃)_{g-3}, or —R(CH₂)C(C[pentose])_{g}(CH₃)_{g-3}, wherein g is an integer: from 1 to 3, inclusive. In a further embodiment, g is an integer from 2 to 3, inclusive.

A peptidyl moiety comprises two or more amino acid residues joined by an amide (peptidyl) linkage between a carboxyl carbon of one amino acid and an amine nitrogen of another. An amino acid is any molecule having a carbon atom bonded to all of (a) a carboxyl carbon (which may be referred to as the “C-terminus”), (b) an amine nitrogen (which may be referred to as the “N-terminus”), (c) a hydrogen, and (d) a hydrogen or an organic moiety. The organic moiety can be termed a “side chain.” The organic moiety can be further bonded to the: amine nitrogen (as in the naturally occurring amino acid proline) or to another atom (such as an atom of the fullerene, among others), but need not be further bonded to any atom. The carboxyl carbon, the amine nitrogen, or both can be bonded to atoms other than those to which they are bonded in naturally-occurring peptides and the amino acid remain an amino acid according to the above description.

Similarly, but independently, in one embodiment X² can be selected from —H; —COOH; —CONH₂, —CONHR′; —CONR′₂; —COOR′; —CHO; —(CH₂)_{d}OH; a peptidyl moiety; a heterocyclic moiety; a branched moiety comprising one or more terminal —OH, —NH₂, triazole, tetrazole, or sugar groups; or a salt thereof. In one embodiment, X² can be selected from —R, —RCOOH, —RCONH₂, —RCONHR′, —RCONR′₂, —RCOOR′, —RCHO, —R(CH₂)_{d}OH, a peptidyl moiety, or a salt thereof. In one embodiment, X² can be selected from —H.; —COOH; —CONH₂; —CONHR′; —CONR′₂; —COOR′; —CHO; —(CH₂)_{d}OH; a peptidyl moiety; —R; —RCOOH; —RCONH₂; —RCONHR′; —RCONR′₂; —RCOOR′; —RCHO; —R(CH₂)_{d}OH.; a heterocyclic moiety; a branched moiety comprising one or more terminal —OH, —NH₂, triazole, tetrazole, or sugar groups; or a salt thereof.

In one embodiment, the substituted fullerene is a decarboxylation product of (C₆₀(>C(COOH)₂)₃)(C3). By “decarboxylation product of C3” is meant the product of a reaction wherein 0 or 1 carboxy (—COOH) groups are removed from each of the three malonate moieties (>C(COOH)₂) of C3 and replaced with —H, provided at least one of the malonate moieties has 1 carboxy group replaced with —H. This can be considered as the loss of CO₂ from a malonate moiety. Decarboxylation can be performed by heating C3 in acid, among other techniques.

During Decarboxylation of C3, only CO₂ loss from C3 is observed; each malonate moiety retains at least one carboxyl; and the Decarboxylation stops at loss of 3 CO₂ groups, one from each malonate moiety of C3. The skilled artisan having the benefit of the present disclosure will recognize that substituted fullerenes having 1, 2, 4, 5, or 6 malonate moieties

In C3, each malonate moiety has a carboxy group pointing to the outside (away from the fullerene), which we herein term exo, and a carboxy group pointing to the inside (toward the fullerene), which we herein term endo. An exemplary chemical structure for C3 is shown below.

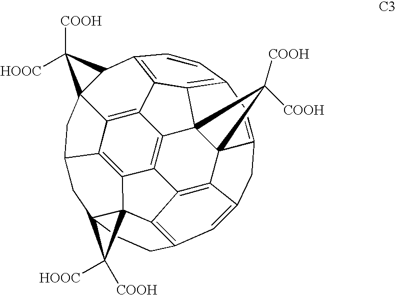

In one embodiment of the present invention, the substituted fullerene comprises a fullerene core (Cₙ), and from 1 to 6 dendrons bonded to the fullerene core.

A dendron within the meaning of this disclosure is an addendum of the fullerene which has a branching at the end as a structural unit. Dendrons can be considered to be derived from a core, wherein the core contains two or more reactive sites. Each reactive site of the core can be considered to have been reacted with a molecule comprising an active site (in this context, a site that reacts with the reactive site of the core) and two or more reactive sites, to define a first generation dendron. First generation dendrons are within the scope of the term “dendron,” as used herein. Higher generation dendrons can be considered to have formed by each reactive site of the first generation dendron having been reacted with the same or another molecule: comprising an active site and two or more reactive sites, to define a second generation dendron, with subsequent generations being considered to have been formed by similar additions to the latest generation. Although dendrons can be formed by the techniques described above, dendrons formed by other techniques are within the scope of “dendron” as used herein.

The core of the dendron is bonded to the fullerene by one or more bonds between (a) one or more carbons of the fullerene and (b) one or more atoms of the core. In one example, the core of the dendron is bonded to the fullerene in such a manner as to form a cyclopropanyl ring.

In one example, the core of the dendron comprises, between the sites of binding to the fullerene and the reactive sites of the core, a spacer that can be a chain of 1 to about 100 atoms, such as about 2 to about 10 carbon atoms.

The generations of the dendron can comprise trivalent or polyvalent elements such as, for example, N, C, P, Si, or polyvalent molecular segments such as aryl or heteroaryl. The number of reactive sites for each generation can be about two or about three. The number of generations can be between 1 and about 10, inclusive.

More information regarding dendrons suitable for adding to fullerenes can be found in Hirsch et al., U.S. Pat. No. 6,506,928, the disclosure of which is hereby incorporated by reference in its entirety.

An exemplary chemical structure for a DF1 is shown below.

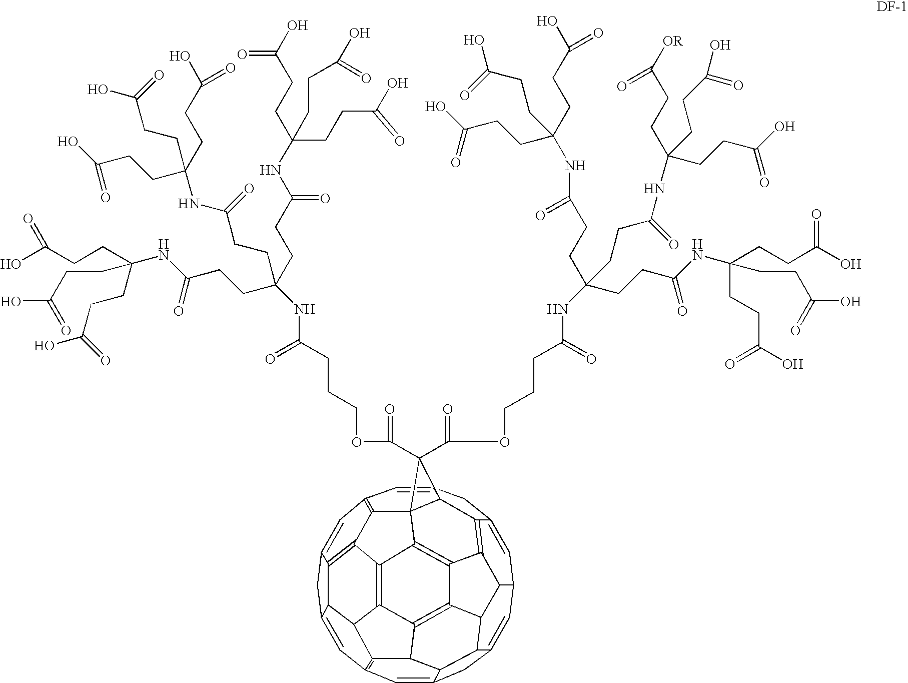

In an example, the disclosed nanoparticle-based anticoagulant includes at least one of a substituted fullerene (e.g., C3, a C3 analog, DF1, a DF1 analog) or a dendrimer such as a PAMAM dendrimer.

As used herein, the dendrimers are unimolecular assemblages that possess three distinguishing architectural features, namely, (a) an initiator core, (b) interior layers (generations, G or Gen) composed of repeating units, radially attached to the initiator core, and (c) an exterior surface of terminal functionality (e.g., terminal functional groups) attached to the outermost generation as disclosed previously in U.S. Pat. No. 5,527,524 which is hereby incorporated by reference in its entirety. The size and shape of a dendrimer molecule and the functional groups present in the dendrimer molecule can be controlled by the choice of the initiator core, the number of generations (e.g., tiers) employed in creating the dendrimer, and the choice of the repeating units employed at each generation. Since the dendrimers can be isolated at any particular generation, a means is provided for obtaining dendrimers having desired properties.

The choice of the dendrimer components affects the properties of the dendrimers. The initiator core type can affect the dendrimer shape, producing (depending on the choice of initiator core), for example, spheroid-shaped dendrimers, cylindrical or rod-shaped dendrimers, ellipsoid-shaped dendrimers, or mushroom-shaped dendrimers. Sequential building of generations (e.g., generation number and the size and nature of the repeating units) determines the dimensions of the dendrimers and the nature of their interior.

Dendrimers that are branched polymers containing dendritic branches having functional groups distributed on the periphery of the branches can be prepared with a variety of properties. For example, dendrimers can be prepared to possess unsymmetrical (unequal segment) branch junctures. Alternatively, dendrimers can be prepared to include symmetrical (equal segment) branch junctures with surface groups, two different interior moieties (represented respectively by X and Z′) with interior void space that varies as a function of the generation (G). Such dendrimers can be advanced through enough generations to totally enclose and contain void space, to give an entity with a predominantly hollow interior and a highly congested surface.

It is the tiered structure that is the essence of the dendrimers rather than the elemental composition. Therefore, the repeating units may be composed of a combination of any elements, so long as these units possess the properties of multiplicity and are assembled into the tiered structure as described herein. These repeat units may be composed entirely of elements that are commonly seen in polymeric structures, such as carbon, hydrogen, oxygen, sulfur, and nitrogen, or may be composed of less traditional elements, provided that these repeat units allow a stable branched structure to be constructed. For example, metalloids and transition metals are well known in the art to form stable covalent compounds and complexes with organic moieties. These stable covalent compounds and complexes with organic moieties can exist as branched materials such as, for example, boranes, borates, germanes, stannanes, and plumbanes, or non-branched linkages such as, for example, dialkyl zincs or mercuries. The use of appropriate ligands can make a transition metal, such as cobalt, function as a branching unit (by connecting three separate ligands) or a non-branched linkage (by connecting two separate ligands). Therefore, branched structures fitting the patterns described herein and incorporating any element are within the scope of the present disclosure.

Also, dendrimers, when advanced through sufficient generations exhibit “dense packing” where the surface of the dendrimer contains sufficient terminal moieties such that the dendrimer surface becomes congested and encloses void spaces within the interior of the dendrimer. This congestion can provide a molecular level barrier which can be used to control diffusion of materials into or out of the interior of the dendrimer.

In an example, surface chemistry of the dendrimers can be controlled in a predetermined fashion by selecting a repeating unit which contains the desired chemical functionality or by chemically modifying all or a portion of the surface functionalities to create new surface functionalities. These surfaces may either be targeted toward specific sites or made to resist uptake by particular organs or cells. In an alternative use of the dendrimers, the dendrimers can themselves be linked together to create polydendritic moieties (“bridged dendrimers”) which are also suitable as carriers.

In addition, the dendrimers can be prepared so as to have deviations from uniform branching in particular generations, thus providing a means of adding discontinuities (e.g., deviations from uniform branching at particular locations within the dendrimer) and different properties to the dendrimer.

In an example, the dendrimer is PAMAM dendrimer. PAMAM dendrimers are based on an ethylenediamine core, and branched units constructed from both methyl acrylate and ethylenediamine. A variety of PAMAM dendrimers are commercially available from Dendritic Nanotechnologies, Inc. (Mt. Pleasant, Mich.) and Aldrich (Milwaukee, Wis.). The half-generations of PAMAM dendrimers, such as NCL22, possess surfaces of carboxylate groups and the full-generations of PAMAM dendrimers possess surfaces of amino groups. NCL22 is a fourth and a half generation (G4.5) PAMAM dendrimer with 64 carboxy end groups on its surface. An exemplary chemical structure for the PAMAM dendrimer NCL22 is provided below.

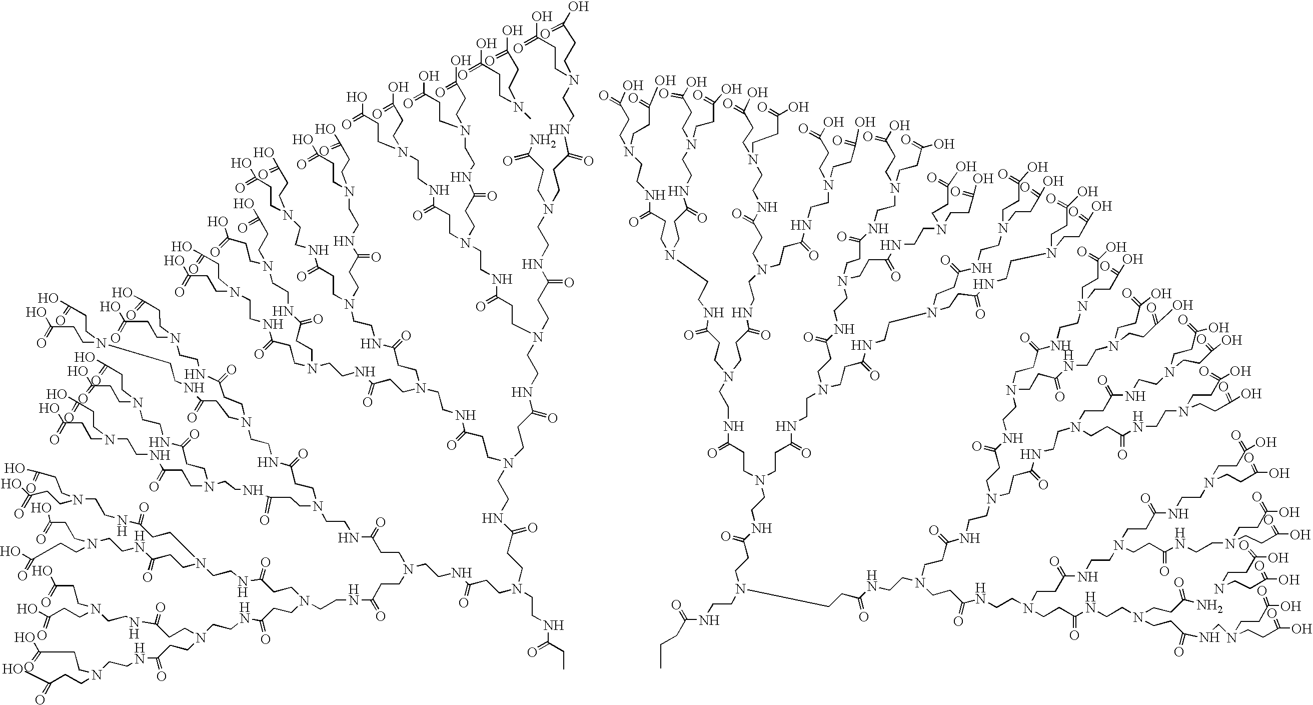

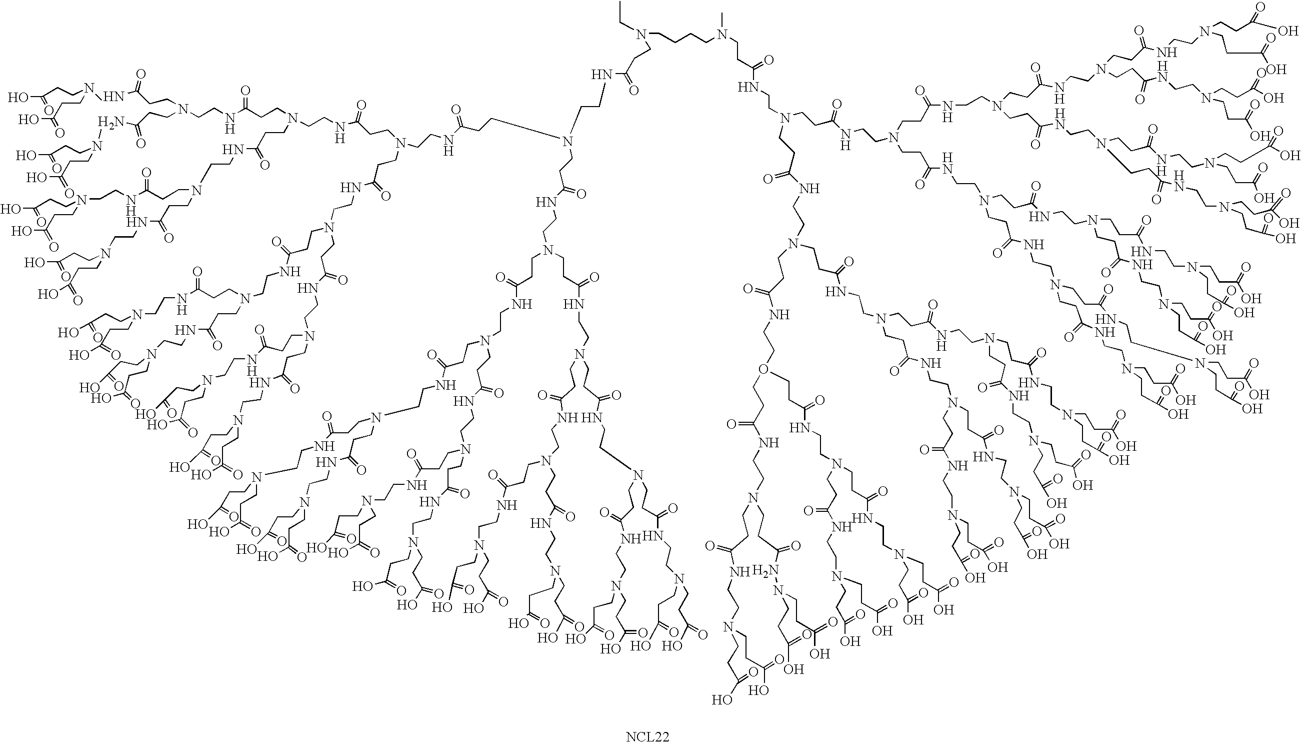

It is contemplated that additional PAMAM dendrimer such as third and a half generation and fourth generation (PAMAM G3.5, PAMAM G4, or PAMAM G4-OH) dendrimers can be employed.

In an additional example, the nanoparticle-based anticoagulant is a nanoparticle that includes a core (for example, a fullerene) and carboxy-terminated dendritic branches. According to a further example, the nanoparticle that includes carboxy-terminated dendritic branches does not also include any other types or classes of branch or substituent groups coupled to the core.

Particular method embodiments contemplate the use of solvates (such as hydrates), pharmaceutically acceptable salts and/or different physical forms of C3, DF1, NCL22 or any of their analogues as herein described below.

1. Solvates, Salts and Physical Forms

“Solvate” means a physical association of a compound with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol associated compound, methanol associated compounds, and the like. “Hydrate” is a solvate wherein the solvent molecule(s) is/are H₂O.

The disclosed compounds also encompass salts including, if several salt-forming groups are present, mixed salts and/or internal salts. The salts are generally pharmaceutically-acceptable salts that are non-toxic. Salts may be of any type (both organic and inorganic), such as fumarates, hydrobromides, hydrochlorides, sulfates and phosphates. In an example, salts include non-metals (e.g., halogens) that form group VII in the periodic table of elements.

Additional examples of salt-forming groups include, but are not limited to, a carboxyl group, a phosphonic acid group or a boronic acid group, that can form salts with suitable bases. These salts can include, for example, nontoxic metal cations which are derived from metals of groups IA, IB, IIA and IIB of the periodic table of the elements. In one embodiment, alkali metal cations such as lithium, sodium or potassium ions, or alkaline earth metal cations such as magnesium or calcium ions can be used. The salt can also be a zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2-hydroxyethyl)amine, or N-methyl-D-glucamine, or quaternary ammonium compounds such as tetrabutylammonium salts.

Particular compounds possess at least one basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid.

Additional counterions for forming pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of a composition.

In certain embodiments the compounds used in the method are provided are polymorphous. As such, the compounds can be provided in two or more physical forms, such as different crystal forms, crystalline, liquid crystalline or non-crystalline (amorphous) forms.

2. Use for the Manufacture of a Medicament

Any of the above described compounds (e.g., C3, a C3 analog, DF1, a DF1 analog, NCL22, a NCL22 analog or a hydrate or pharmaceutically acceptable salt) or combinations thereof are intended for use in the manufacture of a medicament for preventing undesirable platelet aggregation in a subject or for the treatment of a blood clotting disorder such as thrombosis or peripheral arterial occlusion. Formulations suitable for such medicaments, subjects who may benefit from same and other related features are described elsewhere herein.

B. Methods of Synthesis

The disclosed nanoparticle-based anticoagulants including substituted fullerenes and PAMAM dendrimers can be synthesized by various methods. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). In particular, the substituted fullerenes can be synthesized by the methods disclosed by U.S. Pat. No. 6,538,153 (Hirsch et al.), WO 2005/035441 and United States Patent Publication No. 2003/0036562 all of which hereby incorporated by reference in its entirety. Hirsch et al. disclose a method of synthesis of water soluble fullerene polyacids using a macrocyclic malonate reactant. The PAMAM dendrimers can be synthesized by methods disclosed by Tomalia, D. A. & Durst, H. D. (Techniques in Current Chemistry, ed. Weber, E. (Springer, Berlin) 193-245, 2003), Tomalia, D. A. et al. (Angew. Chem. Int. Ed. Engl. 29: 138-175, 1990), U.S. Pat. No. 5,527,524, U.S. Pat. No. 4,587,329, International Patent Publication No. WO2004069878 (Tomalia, D. A. et al.) and International Patent Publication No. WO2005028432 (Tomalia, D. A. et al.) all of which are incorporated by reference in their entireties. In addition, the disclosed compounds are available for research purposes from C Sixty, Inc (Houston, Tex.). Further, PAMAM dendrimers are commercially available from Dendritic Nanotechnologies, Inc. (Mt. Pleasant, Mich.) and Aldrich (Milwaukee, Wis.).

Compounds as described herein may be purified by various methods, including chromatographic means, such as HPLC, preparative thin layer chromatography, flash column chromatography and ion exchange chromatography. Any suitable stationary phase can be used, including normal and reversed phases as well as ionic resins.

### IV. Pharmaceutical Compositions

The disclosed nanoparticle-based anticoagulants including at least one of C3, a C3 analog, DF1, a DF1 analog, NCL22, and a NCL22 analog can be useful, at least, for the treatment of blood clotting disorders such as thrombosis or peripheral arterial occlusion. In addition, such anticoagulants are useful for preventing undesired platelet aggregation that often occurs during implantation of a medical device such as a catheter. Accordingly, pharmaceutical compositions comprising at least one disclosed substituted fullerene or PAMAM dendrimer or analogue thereof are also described herein.

Formulations for pharmaceutical compositions are well known in the art. For example, Remington's Pharmaceutical Sciences, by E. W. Martin, Mack Publishing Co., Easton, Pa., 15th Edition, 1975, describes exemplary formulations (and components thereof) suitable for pharmaceutical delivery of the disclosed nanoparticle-based anticoagulants. Pharmaceutical compositions comprising at least one of these compounds can be formulated for use in human or veterinary medicine. Particular formulations of a disclosed pharmaceutical composition may depend, for example, on the mode of administration (e.g., oral or parenteral) and/or on the disorder to be treated (e.g., thrombosis, peripheral arterial occlusion, or catheter obstruction). In some embodiments, formulations include a pharmaceutically acceptable carrier in addition to at least one active ingredient, such as a nanoparticle-based anticoagulant compound including at least one of a substituted fullerene (such as C3, a C3 analog, DF1, a DF1 analog) or a PAMAM dendrimer (such as NCL22 or NCL22 analog) or combination thereof.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions such as powder, pill, tablet, or capsule forms conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances or excipients, such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

The disclosed pharmaceutical compositions may be formulated as a pharmaceutically acceptable salt. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydriodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

The dosage form of a disclosed pharmaceutical composition will be determined by the mode of administration chosen. For example, in addition to injectable fluids, oral dosage forms may be employed. Oral formulations may be liquid such as syrups, solutions or suspensions or solid such as powders, pills, tablets, or capsules. Methods of preparing such dosage forms are known, or will be apparent, to those skilled in the art.

Certain embodiments of the pharmaceutical compositions comprising a disclosed compound may be formulated in unit dosage form suitable for individual administration of precise dosages. The amount of active ingredient such as C3, a C3 analog, DF1, a DF1 analog, NCL22, a NCL22 analog or combination thereof administered will depend on the subject being treated, the severity of the disorder, and the manner of administration, and is known to those skilled in the art. Within these bounds, the formulation to be administered will contain a quantity of the extracts or compounds disclosed herein in an amount effective to achieve the desired effect in the subject being treated.

### V. Methods of Use

The present disclosure includes methods of treating blood clotting disorders including thrombosis or peripheral arterial occlusion. Disclosed methods include administering a nanoparticle-based anticoagulant such as a substituted fullerene, a PAMAM dendrimer or a combination thereof or (and, optionally, one or more other pharmaceutical agents) to a subject in a pharmaceutically acceptable carrier and in an amount effective to treat the blood clotting disorder. In one example, the substituted fullerene is C3, a C3 analog, DF1, a DF1 analog, or a mixture thereof. In another example, the PAMAM dendrimer is NCL22 or a NCL22 analog. In a further example, the nanoparticle-based anticoagulant is a combination of one or more of the substituted fullerenes and NCL22 or a NCL22 analog.

Routes of administration useful in the disclosed methods include but are not limited to oral and parenteral routes, such as intravenous (iv), intraperitoneal (ip), rectal, ophthalmic, nasal, and transdermal. Formulations for these dosage forms are described above. In addition, routes of administration include the medical device itself.

In an example, a medical device such as a stent or catheter is coated with at least one nanoparticle-based anticoagulant. The nanoparticle-based anticoagulant can include at least one of the disclosed anticoagulant substituted fullerenes (such as C3, a C3 analog, DF1, a DF1 analog, or a mixture thereof), a PAMAM dendrimer (such as NCL22, a NCL22 analog) or a combination thereof. In such example, the medical device can be partially or completely coated with the nanoparticle-based anticoagulant. For instance, the medical device can be partially coated with the nanoparticle-based anticoagulant such as at the points at which the medical device interacts with the subject's vessel, organ or tissue. Such configuration is believed to reduce undesired platelet formation while minimizing the amount of coating material and time required to prepare the device. In a further example, the medical device is substantially coated with one or more of the disclosed nanoparticle-based anticoagulants.

It is contemplated that any chemical or mechanical bond or force, including linking agents can be used to coat the device. For example, a device composed of a first substance can be “coated” with a second substance (e.g., a nanoparticle-based anticoagulant) via a linking agent that is a third substance. Linker binding groups or other appropriate chemical reactive groups can be used to participate in linkage chemistries (derivitization) with linking agents such as, e.g., substituted silanes, diacetylenes, acrylates, acrylamides, vinyl, styryls, silicon oxide, boron oxide, phosphorus oxide, N-(3-aminopropyl)-3-mercapto-benzamide, 3-aminopropyl-trimethoxysilane, 3-mercaptopropyl-trimethoxysilane, 3-maleimidopropyl-trimethoxysilane, 3-hydrazidopropyl-trimethoxysilane, hydroxysuccinimides, maleimides, haloacetyls, pyridyl disulfides, hydrazines, ethyldiethylamino propylcarbodiimide, and/or the like. Alternatively, the coating can be directly linked (tethered) to the first surface through silane groups.

In a further example, a medical device such as a stent or catheter is impregnated with at least one nanoparticle-based anticoagulant. For example, the nanoparticle-based anticoagulant can include at least one of an anticoagulant substituted fullerene (such as C3, a C3 analog, DF1, a DF1 analog, or a mixture thereof), a PAMAM dendrimer (such as NCL22 or a NCL22 analog) or a combination thereof. In such example, the medical device is at least partially impregnated with the anticoagulant so that at least one surface including the nanoparticle-based anticoagulant enhances the interaction of the device with the organ, tissue, vessel, and the like in which the device is used. For instance, the surfaces are employed to improve biointegration of the device into the subject's body by inhibiting platelet aggregation and thus, thrombosis at the implantation site. In another example, the nanostructured components (e.g., nanoparticle-based anticoagulant) are substantially impregnated throughout the device so that the multiple surfaces (such as the outer and inner surfaces) of the medical device include the nanoparticle-based anticoagulants.

It is contemplated that the medical device can be coated or impregnated with materials in addition to the disclosed nanoparticle-based anticoagulants to further enhance their bio-utility. Examples of suitable coatings are medicated coatings, drug-eluting coatings, hydrophilic coatings, and smoothing coatings. For example, there are known in the art drug eluting coronary stents, such as the U.S. FDA-approved Cordis Cypher™ sirolimus-eluting stent and the Boston Scientific Taxus™ paclitaxel-eluting stent system. This new therapy involves coating the outer aspect of a standard coronary stent with a thin polymer containing medication that can prevent the formation of scar tissue at the site of coronary intervention. Examples of the medications on the currently available stents are sirolimus and paclitaxel, as well as anti-inflammatory immunomodulators such as Dexamethasone, M-prednisolone, Interferon, Leflunomide, Tacrolimus, Mizoribine, statins, Cyclosporine, Tranilast, and Biorest; antiproliferative compounds such as Taxol, Methotrexate, Actinomycin, Angiopeptin, Vincristine, Mitomycin, RestenASE, and PCNA ribozyme; migration inhibitors such as Batimastat, Prolyl hydroxylase inhibitors, Halofuginone, C-proteinase inhibitors, and Probucol; and compounds which promote healing and re-endothelialization such as VEGF, Estradiols, antibodies, NO donors, BCP671, and the like. Sirolimus, for example, had been used previously to prevent rejection following organ transplantation. The use of polymer coatings on stents often leads to thrombosis and as a result, requires administration of an anticoagulant for extended periods of time (e.g., three months after placement of the device). The use of polymer coatings in addition the coating or impregnation of the medical device such as a stent with the disclosed nanoparticle-based anticoagulants can alleviate the previously reported thrombosis.

An effective amount of the nanoparticle-based anticoagulant will depend, at least, on the particular method of use, the subject being treated, the severity of the disorder, and the manner of administration of the therapeutic composition. A “therapeutically effective amount” of a composition is a quantity of a specified compound sufficient to achieve a desired effect in a subject being treated. For example, this may be the amount of a nanoparticle-based anticoagulant necessary to prevent, inhibit, reduce or relieve the blood clotting disorder such as thrombosis and/or one or more symptoms of the disorder such as undesired platelet aggregation in a subject. Ideally, a therapeutically effective amount of the nanoparticle-based anticoagulant is an amount sufficient to prevent, inhibit, reduce or relieve the blood clotting disorder and/or one or more symptoms such as catheter obstruction caused by the disorder without causing a substantial cytotoxic effect on host cells.

Therapeutically effective doses of a disclosed nanoparticle-based anticoagulant compound or pharmaceutical composition can be determined by one of skill in the art. An example of a dosage range is from about 0.001 to about 10 mg/kg body weight orally in single or divided doses. In particular examples, a dosage range is from about 0.005 to about 5 mg/kg body weight orally in single or divided doses (assuming an average body weight of approximately 70 kg; values adjusted accordingly for persons weighing more or less than average). For oral administration, the compositions are, for example, provided in the form of a tablet containing from about 1.0 to about 50 mg of the active ingredient, particularly about 2.5 mg, about 5 mg, about 10 mg, or about 50 mg of the active ingredient for the symptomatic adjustment of the dosage to the subject being treated. In one exemplary oral dosage regimen, a tablet containing from about 1 mg to about 50 mg active ingredient is administered three to four times a day.

The specific dose level and frequency of dosage for any particular subject may be varied and will depend upon a variety of factors, including the activity of the specific compound, the metabolic stability and length of action of that compound, the age, body weight, general health, sex and diet of the subject, mode and time of administration, rate of excretion, drug combination, and severity of the condition of the subject undergoing therapy. In one example, the nanoparticle-based anticoagulant is administered in conjunction with insertion of an in-dwelling device into the subject. For example, the nanoparticle-based anticoagulant is administered locally at an implantation site of the in-dwelling device.

### VI. Kits/Systems

In some examples, the disclosure provides kits for practice of the methods described herein and which optionally comprise the substrates of the disclosure. In various embodiments, such kits comprise one or more devices such as a catheter and any necessary reagents, apparatuses, and materials used to fabricate and/or use such a device. For example, a kit can include one or more catheters and a coating compound that includes one or more nanoparticle-based anticoagulants. In another example, the kit can include one or more medical devices and one or nanoparticle-based anticoagulants that can be administered to the subject at the time of device implantation.

In addition, the kits can optionally include instructional materials containing directions (e.g., protocols) for coating the medical device surface prior to insertion of the device. Preferred instructional materials give protocols for utilizing the kit contents. The instructional materials optionally include written instructions (e.g., on paper, on electronic media such as a computer readable diskette, CD or DVD, or access to an internet website giving such instructions) for the application of the nanoparticle-based anticoagulant to the medical device or administration of the anticoagulant at the time of implantation of the device.

The following examples are provided to illustrate certain particular features and/or embodiments. These examples should not be construed to limit the invention to the particular features or embodiments described.

### EXAMPLES

### Example 1

### Methods for Hemolysis Assay, CFU-GM Assay, and Platelet Aggregation Assay

This example provides general methods for analysis of hemolysis, myelosuppression, and platelet aggregation. All of the following procedures are described in detail at http://ncl.cancer.gov/working_assay-cascade.asp which is hereby incorporated by reference as of Oct. 9, 2006.

Hemolysis Assay. This assay employs a protocol for quantitative colorimetric determination of hemoglobin in whole blood (total blood hemoglobin—TBH) and hemoglobin released into plasma (plasma free hemoglobin—PFH) when blood was exposed to nanoparticles. In brief, hemoglobin and its derivatives, except sulfhemoglobin, were oxidized to methemoglobin by ferricyanide in the presence of alkali. Cyanmethemoglobin (CMH) was formed from the methemoglobin by its reaction with cyanide (Drabkin's solution) and then detected by spectrophotometer set at 540 nm. The hemoglobin standard was used to build a standard curve covering the concentration range from 0.025 to 0.80 mg/mL and to prepare quality control samples at low (0.0625 mg/mL), mid (0.125 mg/mL) and high (0.625 mg/mL) concentrations to monitor assay performance. Required sample volume was 300 μL, e.g., 100 μL per test-replicate. The results expressed as percent of hemolysis were used to evaluate the acute in vitro hemolytic properties of nanoparticles.

The following reagents were used in the assay: cyanmethemoglobin (CMH) reagent, (StanBio, cat. # 0321-380); hemoglobin standard (StanBio, cat. # 0325-006); Ca/Mg free DPBS (Sigma, catalogue no: D8537); pooled normal human whole blood anti-coagulated with Li-heparin; poly-L-Lysine hydrobromide (MW 150 000-300 000, Sigma, cat #P1399); polyethylene glycol (av. MW 8 000, Sigma cat # P1458); and distilled water. It is contemplated that equivalent reagents from other vendors can be used.

Table 1 provides an example of preparation of calibrations standards.

| | Nominal Conc. | |
|---|---|---|
| Level | (mg/mL) | Preparation Procedure |
| Cal 1 | 0.80 | 2 mL of stock solution |
| Cal 2 | 0.40 | 1 mL Cal1 + 1 mL CMH reagent |
| Cal 3 | 0.20 | 1 mL Cal3 + 1 mL CMH reagent |
| Cal 4 | 0.10 | 1 mL Cal3 + 1 mL CMH reagent |
| Cal 5 | 0.05 | 1 mL Cal4 + 1 mL CMH reagent |
| Cal 6 | 0.025 | 1 mL Cal5 + 1 mL CMH reagent |

The positive control was prepared by dissolving poly-L-Lysine powder to a final concentration of 1% (10 mg/mL) in sterile distilled water. Daily use aliquots were prepared and stored at a nominal temperature of −20° C. Alternatively, 1% TritonX-100 in water was used as a positive control. PLL resulted in 30±5% hemolysis. Triton X-100 resulted in 90±5% hemolysis. Polyethylene glycol supplied as a 40% stock solution in water was used as the negative control. The stock solution was stored at a nominal temperature of +4° C.

For the initial screen, the test concentration was selected based on results from general toxicity assays. A nanoparticle that revealed toxicity in general toxicity assay was tested at two concentrations selected at the low and the high end of the dose response curve. A nanoparticle that did not reveal toxicity in a general toxicity assays was tested at one concentration equal to highest dose tested in general toxicity assay. The assay required 300 μL of test material. Nanoparticle and buffer used for its storage/reconstitution were tested in the same assay. Respectively, 300 μL of the buffer is required.

Whole blood was collected in tubes containing Li-heparin as anti coagulant from at least three donors. The blood was stored at 2-8° C. for up to 48 h. On the day of assay, pooled blood was prepared by mixing equal proportion of blood from each donor. Two to three mL aliquot of the pooled blood centrifuged for 15 min at 800 g. The supernatant was collected and kept at room temperature while preparing standard curve, quality controls and total hemoglobin samples. The collected sample was used to determine plasmafree hemoglobin (PFH). Two hundred μL of each calibration standard, quality control and blank cyanmethemoglobin (CMH) reagent was added to each well on the 96 well plate. Two wells were filled for each calibrator and 4 wells for each quality control (QC) and blank. The test samples were positioned so that they were bracketed by the QC. 6.5. Two hundred μL of total blood hemoglobin (TBH) sample prepared was added by combining 20 μL of the pooled whole blood and 5.0 mL of cyanmethemoglobin reagent. Six wells were filled. One hundred μL of plasma was added per well on 96 well plate. Six wells were filled. Then, 100 μL of cyanmethemoglobin reagent was added to each well containing sample. Cyanmethemoglobin reagent were not added to wells containing calibration standards and quality controls. The plate was covered with a plate sealer and gently rocked on a plate shaker for 1-2 minutes (shaker speed settings was vigorous enough to allow mixing the reagent, but to avoid spillage and cross-well contamination; e.g., LabLine shaker speed 2-3). The plate was then read by use of a plate reader at an absorbance of 540 nm to determine hemoglobin concentration. The dilution factor 2 was used for PFH sample and the dilution factor 251 for TBH. If calculated PFH concentration was below 1 mg/mL pooled whole blood was diluted with Ca²⁺/Mg²⁺ free DPBS to adjust total hemoglobin concentration to 10±2 mg/mL. In an eppendorf tube, 100 μL of sample, blank (e.g., buffer used to reconstitute test sample), positive or negative control was added. Six tubes for each unknown sample; 3 tubes for the blank, 2 tubes for the positive control and 2 tubes for the negative control were prepared. If sample volume was below 100 μL, volume was adjusted with Ca²⁺/Mg²⁺ free DPBS. Seven hundred μL of Ca²⁺/Mg²⁺ free DPBS was then added to each tube. One hundred μL of the prepared whole blood was added to each tube, except for 3 tubes of each test sample. In such tubes, 100 μL of Ca²⁺/Mg²⁺ free DPBS was added instead of the whole blood. These samples represented a “minus blood” control and were used to evaluate potential interference of nanoparticle with the assay (e.g., absorbance at or close to 540 nm, reactivity with CMH reagent etc.) Tubes were covered and gently rotated to mix. Vortexing may damage erythrocytes and therefore was avoided.

Tubes were placed in a water bath set at 37° C. and incubated for 3 hours±15 min mixing the samples every 30 min. Alternatively, tubes were incubated on a tube rotator in an incubator set at 37° C. At the appropriate time, the tubes were removed from the water bath or incubator. If a water bath was used, excess of water was dried with absorbent paper. Tubes were centrifuged for 15 min at 800 g. If nanoparticles have absorbance at or close to 540 nm, removal of these particles from supernatant was required before proceeding to the next step. For example, 10-50 nm colloidal gold nanoparticles have absorbance at 535 nm. Thus, after centrifugation supernatants were transferred to fresh tubes and centrifuged for an additional 30 min at 18 000 g. Method of nanoparticles removal from supernatant is nanoparticles specific, and when applied appropriate validation experiments should be conducted to ensure that a given separation procedure does not affect assay performance. In certain cases removal of particles was not feasible. When this was the case, assay results obtained for a particle incubated with blood was adjusted by subtracting results obtained for the same particle in “minus blood” control and a fresh set of calibrators and quality controls were prepared. Further, to a fresh 96 well plate 200 μL of blank reagent, calibrators, quality controls or total blood hemoglobin sample (TBHd) prepared was added by combining 400 μL of diluted pooled whole blood with Ca²⁺/Mg²⁺ free DPBS to adjust total hemoglobin concentration to 10±2 mg/mL with 5.0 mL of CMH reagent. Two wells were filled for each calibrator, 4 wells for blank and each quality control, and 6 wells for TBHd sample. As before, all test samples were positioned between quality controls on the plate. One hundred μL was added per well of test samples, positive and negative controls. Twelve wells for each sample and 4 wells for each control were filled. One hundred μL of cyanmethemoglobin reagent was added to each well containing sample and controls. Cyanmethemoglobin reagent was not added to wells containing calibration standards, quality controls and TBHd. The plate was covered with a plate sealer and gently mixed on a plate shaker (LabLine shaker speed settings 2-3 or as appropriate for a given shaker). The plate was then read by a plate reader at absorbance of 540 nm to determine concentration of hemoglobin. The dilution factor 16 was used for samples and controls while a dilution factor of 13.5 was used for TBHd.

A four-parameter regression algorithm was used to build calibration curve. The following parameters were calculated for each calibrator and quality control sample: Percent Coefficient of Variation: % CV=SD/Mean×100%. Percent Difference From Theoretical: PDFT=(Calculated Concentration−Theoretical Concentration)×100% Theoretical Concentration % CV for each blank, positive control, negative control and unknown sample in which % CV and PDFT for each calibration standard and quality control was within 20%. The exception was Cal 6, for which 30% was acceptable. A plate was accepted if two-thirds of all QC levels and at least one of each level have demonstrated acceptable performance. If not, the entire run was repeated. % CV for each positive control, negative control and unknown sample were within 20%. At least one replicate of positive and negative control were acceptable for run to be accepted. If both replicates of positive control or negative control failed to meet acceptance criterion described above, the run was repeated. Within the acceptable run if two of three replicates of unknown sample failed to meet the described acceptance criterion, this unknown sample was re-analyzed.

CFU-GM Assay. The granulocyte-macrophage colony-forming units (CFU-GM) assay employed murine bone marrow (BM). Hematopoietic stem cells of BM proliferate and differentiate to form discrete cell clusters or colonies. The BM cells were isolated from 8-12 week old mice and cultured in methylcellulose-based medium supplemented with cytokines (mSCF, mIL-3 and hIL-6) either untreated (baseline) or treated with nanoparticles (test). These cytokines promoted formation of granulocyte and macrophage (CFU-GM) colonies. After twelve days of incubation at 37° C. in the presence of 5% CO₂ and 95% humidity number of colonies was quantified in baseline and test samples. A percent of CFU inhibition was then calculated for each test sample. The basic protocol for BM isolation and culture was adopted from technical manual # 28405 developed by StemCell Technologies Inc. The assay required 450 μL of a test-nanoparticles, MethoCult medium (StemCell Technologies Inc cat. # 03534), Fetal Bovine Serum prescreened for hematopoietic stem cells (StemCell Technologies Inc. cat. # 06200), Iscove's MDM with 2% FBS (StemCell Technologies Inc cat. # 07700), sterile distilled water, blunt-end 16 gauge needles (StemCell Technologies Inc cat. # 03534 2.6), cisplatin (positive control; Sigma cat #P4394), Sterile Ca²⁺/Mg²⁺-free DPBS (negative control; Sigma D8537). Equivalent reagents from other vendors can be used.

The MethoCult medium was allowed to thaw at room temperature or in a refrigerator overnight. Once thawed, the medium was vortexed to mix the ingredients thoroughly and then left at a room temperature for approximately 5 minutes to allow air bubbles to dissipate. A 16 gauge blunt-end needle was used to dispense 3 mL of the MethoCult medium into sterile 15 mL tube. The aliquoted medium was stored at a nominal temperature of −20° C. Before the test, aliquots were thawed at room temperature for approximately 20 minutes and kept on ice prior to use. Cisplatin was reconstituted from the lyophilized powder by adding appropriate amount of DMSO to make a stock solution with nominal concentration of 50 mM. Small aliquots were prepared and stored at a nominal temperature of −80° C. Prior to use in the assay, an aliquot of cisplatin was thawed at room temperature and diluted in IMEM supplemented with 2% fetal bovine serum (FBS) to bring the concentration to 2 mM. One hundred fifty (150) μL of this intermediate solution is then added to 3 mL of culture medium. Final concentration of cisplatin in the positive control sample was 50 μM.

The CFU-GM assay required 450 μL of nanoparticles, e.g., three 150 μL samples, each of which is analyzed in duplicate. The following criteria were considered when selecting the concentration of the nanoparticles: i) solubility of nanoparticles in a biocompatible buffer; ii) pH within physiological range; iii) availability of nanomaterial, and iv) stability. For the initial screen the test concentration was selected based on results from general toxicity assays. A nanoparticle that revealed toxicity in general toxicity assays was tested at two concentrations selected at the low and the high end of the dose response curve. A nanoparticle that did not reveal toxicity in a general toxicity assays was tested at one concentration equal to highest dose tested in general toxicity assay.

Bone marrow was isolated from 8-12 weeks old C56BL6 male or female mice. Using a 3 cc syringe with 21 or 22 gauge needle, up to 1-3 mL of cold Iscove's MDM supplemented with 2% FBS was drawn up into the needle. The bevel of needle was then inserted into the marrow shaft and marrow was flushed into a 15 mL tube. This procedure was repeated for tibia and femur. The bone shallow appeared white once all the marrow had been expelled. Keeping the needle below medium surface, medium with cells was drawn up and down with 3 cc syringe and 21 gauge needle 3-4 times to make a single cell suspension. Cells were kept in medium on ice until use. A nucleated cell count was performed by first diluting the cells with 3% acetic acid with methylene blue 1:100 (e.g., 10 μL cells+990 μL 3% acetic acid/methylene blue) and counting the cells by use of either a hemocytometer or automatic cell counter. An average cell count was expected to be 1-2×10⁷ for femur and 0.6-1×10⁷ for tibia. If cell viability (at least 90%) and count were acceptable, MethoCult medium was thawed at room temperature or in refrigerator overnight. Once thawed, tubes were vortexed to ensure all components were thoroughly mixed. Isolated cells were diluted with Iscove's medium supplemented with 2% FBS to 4×10⁵ cells per mL. One hundred and fifty microliters of cell suspension and 150 μL of either Iscove's medium with 2% FBS (baseline), PBS (negative control), Cisplatin (positive control), or nanoparticles (test sample) were added to 3 mL of MethoCult medium. Tubes were vortexed to ensure all cells and medium components are mixed thoroughly and then let to stand for 5 minutes to allow bubbles to dissipate. A 16 gauge blunt-ended needle was attached to a 3 cc syringe, the needle was placed below the surface of solution and drawn up approximately 1 mL. The plunger was gently depressed, expelling the medium completely. This process was repeated until no air space was visible. MethoCult medium was then drawn up with cells into the syringe and 1.1 mL was dispensed per 35 mm dish. All samples were tested in duplicate (N=2). Two duplicates were analyzed for each nanoparticle. The medium was distributed evenly by gently tilting and rotating each dish. 8.11. Two (2) covered dishes with cells and one (1) uncovered dish filled with 3 mL of sterile water were placed into 150 mm and cultured in an incubator maintained at 37° C., 5% CO2 and 95% humidity for 12 days. On the 12^{th} day, dishes were removed from the incubator, colonies were identify and counted as described below. Representative values of CFU-GM for C57BL6 mice at 8-12 weeks of age is 64±16.9.

CFU-GM included CFU-granulocyte (CFU-G), CFU-macrophage (CFU-M) and CFU-granulocyte macrophage (CFU-GM). The colonies contained 30 to thousands of CFU-G, CFU-M or both cell types (CFU-GM). Each colony included at least 30 cells. CFU-GM colonies often contained multiple clusters and appeared as a dense core surrounded by cells. The monocytic lineage cells were large cells with an oval to round shape and appeared to have a drainy or grey center. The granulocytic lineage cells were round, bright, and were much smaller and more uniform in size than macrophages. A Percent Coefficient of Variation was calculated for each control or test according to the following formula: % CV=SD/Mean×100%. A Percent CFU Inhibition was calculated as follows: % CFU-Inhibition=(Baseline CFU-GM−Test CFU-GM)×100% Baseline CFU-GM. Baseline refers to the assay negative control. % CV for each control and test sample was less than 30%. If positive control or negative control failed to meet acceptance criterion, the assay was repeated. Within the acceptable assay, if two of three replicates of unknown sample failed to meet acceptance criterion, the unknown sample was re-analyzed. If two duplicates of the same study sample demonstrated results different more then 30%, the sample was reanalyzed.

Platelet Aggregation Assay. Test nanoparticles were reconstituted in RPMI or other medium that does not interfere with platelet aggregation. The concentration of the nanoparticle was determined by considering the following parameters: i) the solubility of the nanoparticle in a biocompatible buffer; ii) maintaining the pH within a physiological range (pH 7±0.5); iii) availability of nanoparticle; and iv) stability of the nanoparticle. For the initial screen, the test concentration was selected based on results from in vitro toxicity assays. A nanoparticle that revealed toxicity in general toxicity assays, was tested at two concentrations selected at the low and high end of the given dose response curve. A nanoparticle that did not reveal toxicity in a general toxicity assay was tested at one concentration equal to the highest does tested in the general toxicity assay. The assay required 150 μL of test material.

Platelet-rich plasma (PRP) was obtained from fresh pooled human whole blood by spinning freshly drawn blood for 8 minutes at 200 g. PRP was then pooled from at least 3 different donors. It is to be noted that during phlebotomy, the first 2 mLs of blood was discarded. Further, PRP was prepared as soon as possible from the time of obtaining the blood sample and no longer than 1 hour after blood collection. PRP was kept at room temperature and was used within 4 hours of its isolation. Exposure of either the blood or PRP to cold temperatures (<20° C.) was avoided because such temperatures induce platelet aggregations.

Three test tubes for the test sample, two tubes for the positive control and two tubes for the negative control were prepared. Twenty-five microliters of the test sample, positive control or negative control were added to the respective tubes. These samples provided data on the ability of the test nanoparticle to induced platelet aggregation. A second set of tubes were prepared for test-particles plus collagen. These set of tubes provided data on the ability of test-particles to interfere with platelet aggregation caused by collagen. For this set of studies, 25 μL of the positive control and 25 μL of the test nanoparticle was added to each “test-particles plus collagen” tube. Further, 50 μL of the negative control was added to each negative control tube and 25 μL of the positive control and 25 μL of RPMI was added to each positive control tube. In addition, one control tube with 100 μL of phosphate buffer saline (PBS) or RPMI and 25 μL of the nanoparticles was prepared to determine any potential particle interference with instrument counting procedure.

PRP was added to the appropriate tubes, samples were briefly vortexed and then incubated with control or test sample for 15 minutes at a nominal temperature of 37° C. After the incubation period, 10 mL of Isoton II diluent was added into blood cell counter vials. Two vials were prepared for each sample replicate. Twenty microliters of PRP treated with positive control, negative control or test-nanoparticle was then added to the each vial. Vials were covered and gently inverted to mix the diluted samples. PRP was analyzed by a Z2 Particle Count and Size Analyzer (Beckman Coulter) to determine the number of active platelets. Percent Coefficient of Variation (% CV) was equal to the standard deviation divided by the mean multiplied by 100. Platelet count=(5×RC)/100=# platelets×10⁹/L. Further, percent platelet aggregation (% aggregation)=Control Platelet Count−Sample Platelet Count/Control Platelet Count×100). The percent coefficient of variation for each control and test sample was within 25%. If both replicates of the positive or negative control failed to fall within the 25% coefficient of variation, then the run was repeated. Percent platelet aggregation above 20% was considered to be positive (for example, test-particle induces platelet aggregation).

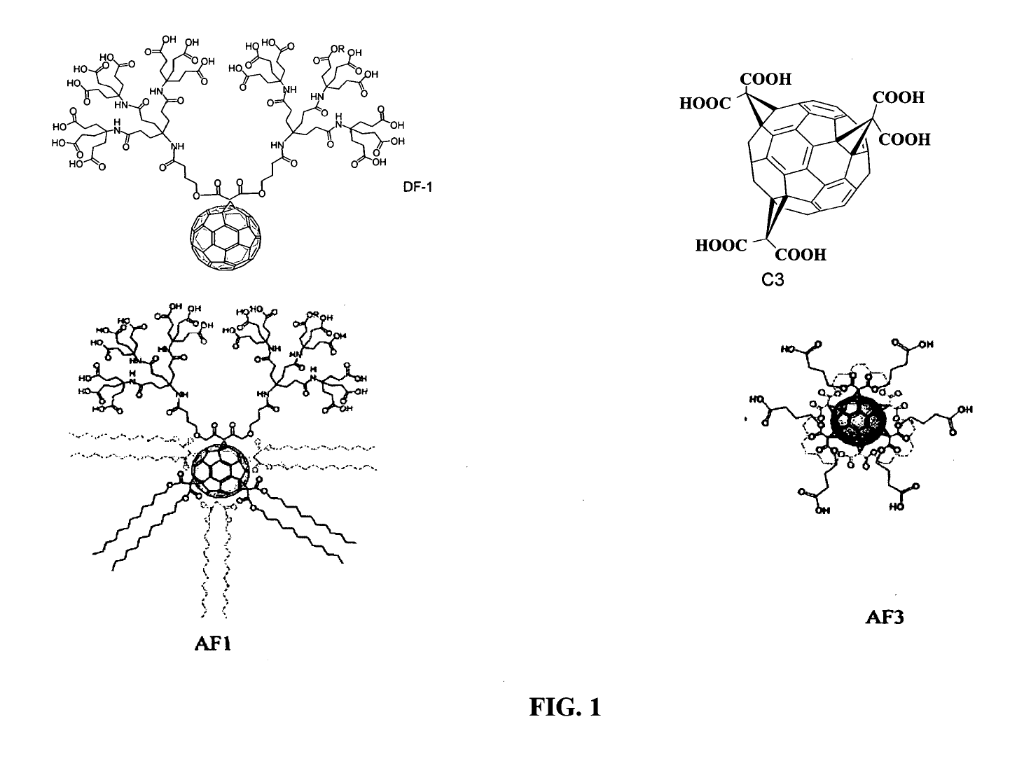

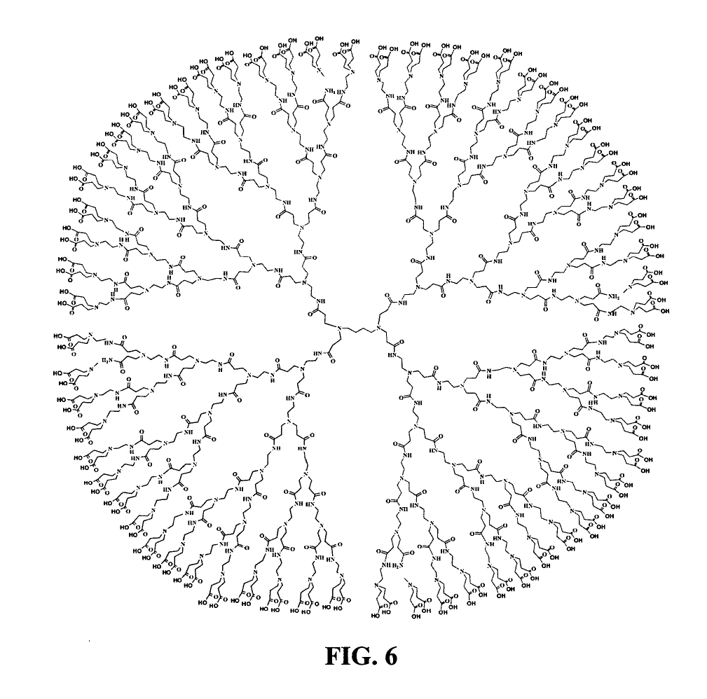

Provider of substituted fullerenes and PAMAM dendrimer. All substituted fullerenes (AF1, AF3, C3, and DF1) and the PAMAM dendrimer NCL22 tested were obtained by C Sixty, Inc (Houston, Tex.). FIG. 1 provides a schematic representation of substituted C₆₀ compounds including C3, DF1, AF1 and AF3 whereas FIG. 6 includes a schematic representation of the carboxy-terminated PAMAM dendrimer NCL22.

### Example 2

### Hemolytic Activity of Substituted C₆₀ Compounds

This example shows the hemolytic activity of substituted C₆₀ compounds C3, DF1, AF1 and AF3.

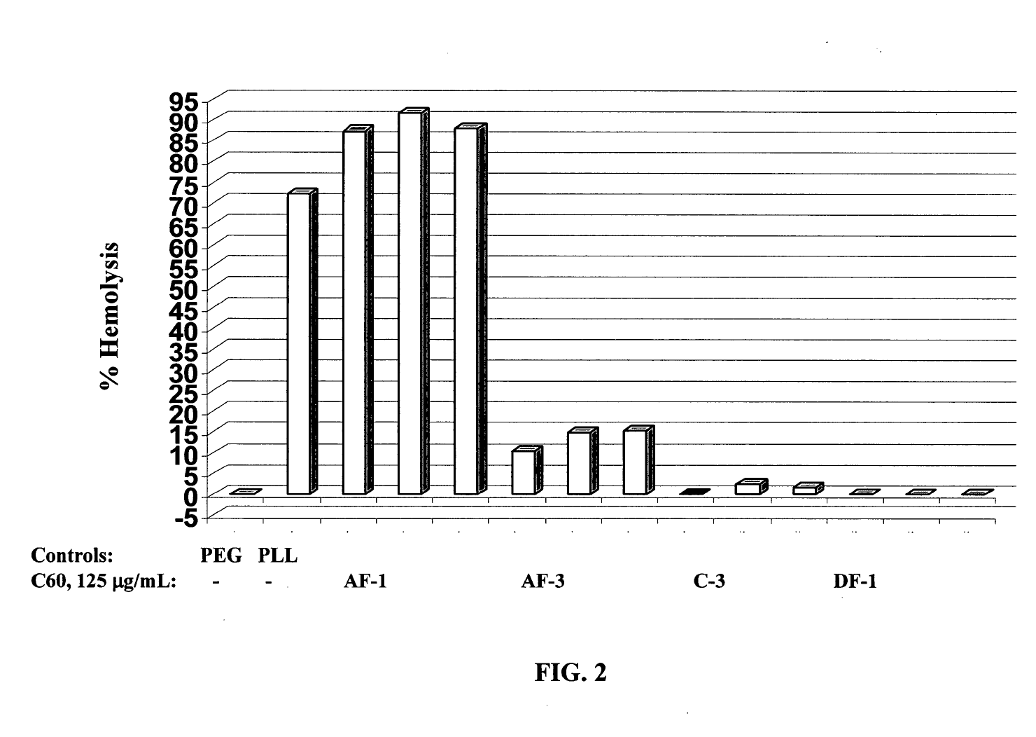

The effect of substituted fullerenes C3, DF1, AF1 and AF3 on hemolysis was determined by use of the hemolysis assay described in detail in Example 1. FIG. 1 provides the exemplary chemical structures of C3, DF1, AF1 and AF3. As illustrated in FIG. 2, AF1 (125 μg/mL) and AF3 (125 μg/mL) demonstrated strong hemolytic activity in which AF1 induced greater than 85% hemolysis. AF3 induced greater than 10% hemolysis. In contrast, substituted fullerenes C3 and DF1 demonstrated relatively no hemolytic activity in which the percent of hemolysis was less than 2%. These studies suggest that C3 and DF1 possess relatively no hemolytic activity and therefore, do not damage erythrocytes at the concentrations tested.

### Example 3

### Myelosuppressive Activity of Substituted C₆₀ Compounds

This example illustrates the lack of myelosuppressive activity of the substituted C₆₀ compounds C3, DF1, AF1 and AF3.

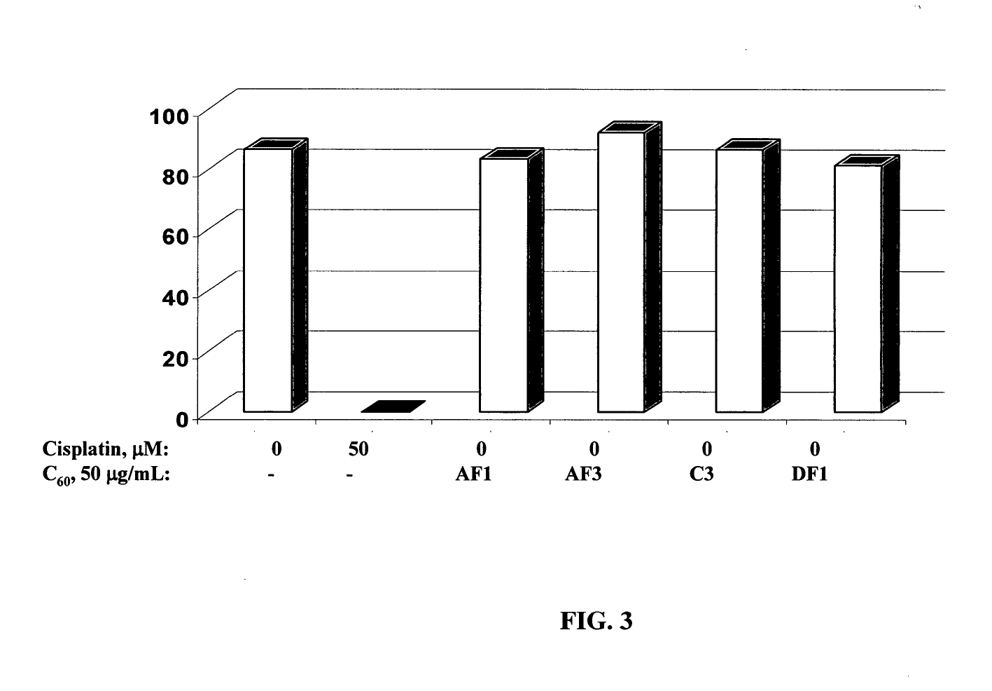

The effect of substituted fullerenes C3, DF1, AF1 and AF3 on myelosuppression was evaluated by use of the CFU-GM assay described in detail in Example 1. As illustrated in FIG. 3, none of the tested substituted fullerenes had a myelosuppressive effect at 50 μg/mL as compared to the positive control, cisplatin (50 μM). In addition, none of the tested substituted fullerenes were able to protect the bone marrow cells from the myelosuppression caused by cisplatin treatment. As shown in FIG. 3, the myelosuppressive effect of cisplatin was independent of the presence of the substituted fullerene. These studies demonstrate that C3, DF1, AF1 and AF3 are not toxic to bone marrow at least at the concentrations tested.

### Example 4

### Effect of Substituted C₆₀ Compounds on Platelet Aggregation

This example illustrates the effect of the substituted C₆₀ compounds C3, DF1, AF1 and AF3 on platelet aggregation in the presence and absence of collagen.

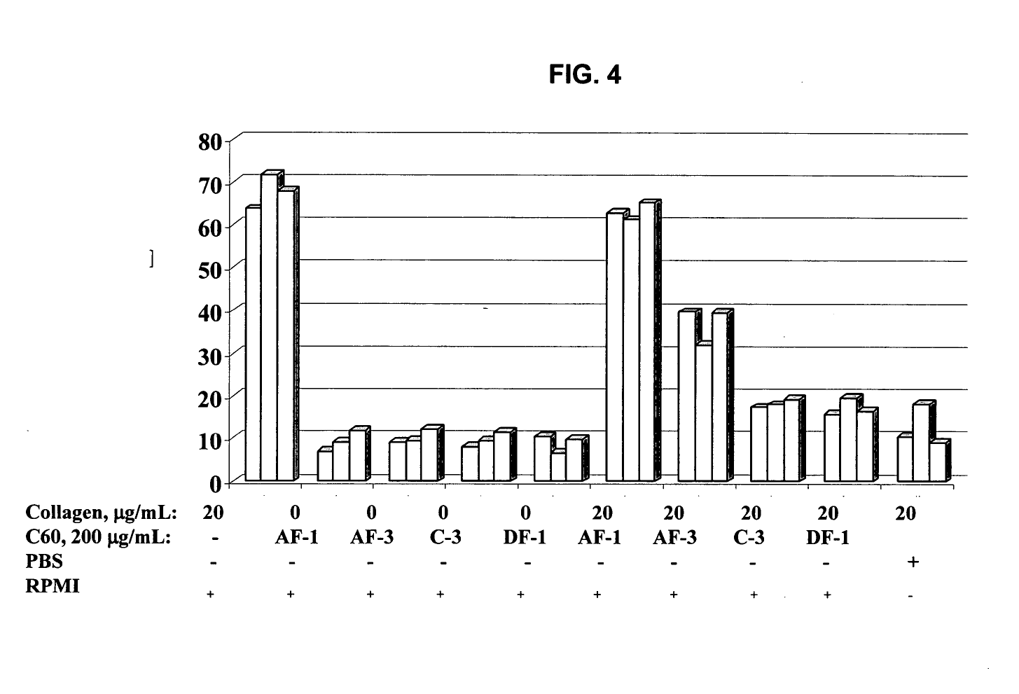

The effect of substituted fullerenes C3, DF1, AF1 and AF3 on platelet aggregation was evaluated by use of the platelet aggregation assay described in detail in Example 1. As illustrated in FIG. 4, collagen (20 μg/mL) induced greater than 60% platelet aggregation. In comparison, C3, DF1, AF1 and AF3 induced on average less than 10% platelet aggregation. However, C3 (200 μg/mL) or DF1 (200 μg/mL) inhibited collagen-induced platelet aggregation. As shown in FIG. 4, C3 or DF1 reduced collagen-induced platelet aggregation from greater than 60% to approximately 15%. AF1 had no effect on collagen-induced aggregation whereas AF3 caused a moderate reduction in collagen-induced platelet aggregation (from 60% to approximately 35%). These studies suggest that C3 and DF1 possess anticoagulant properties in that such compounds inhibit collagen-induced platelet aggregation.

### Example 5

### Effect of Aspirin on Collagen-Induced Platelet Activity

This example illustrates the effect of aspirin on collagen-induced platelet aggregation.

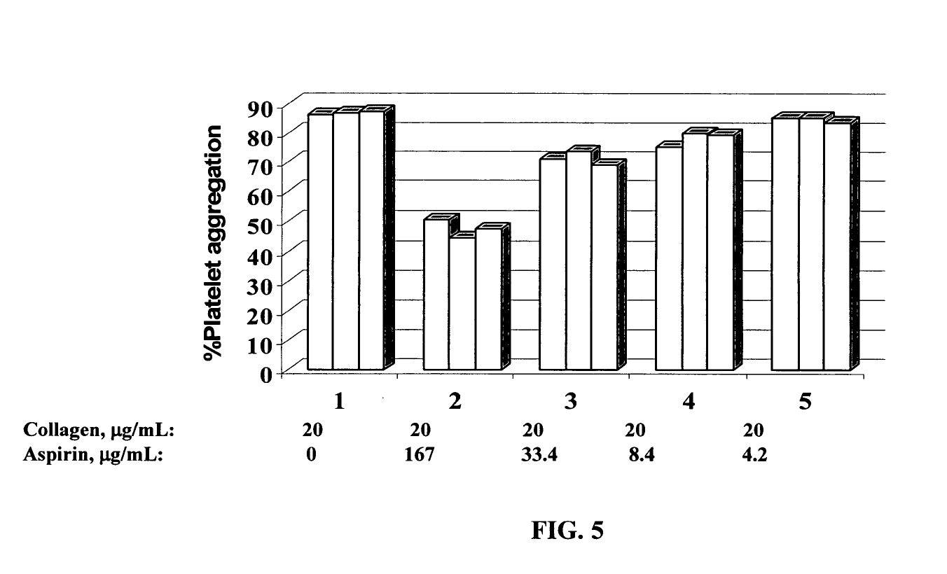

The effect of a known anticoagulant, aspirin on collagen-induced platelet aggregation was determined by use of the platelet aggregation assay described in detail in Example 1. As illustrated in FIG. 5, 25 μg/mL of collagen induced greater than 80% platelet aggregation. This aggregation induced by collagen was inhibited by aspirin. For example, the addition of 167 μg/mL of aspirin resulted in a significant reduction in collagen-induced platelet aggregation (2) as compared to platelet aggregation in the presence of collagen alone (1). Further, the inhibition of collagen-induced platelet aggregation was dose dependent. The greatest amount of inhibition was observed in the presence of 167 μg/mL of aspirin (2). As the concentration of aspirin decreased (from 167 μg/mL (2) to 33.4 μg/mL (3) and then to 16.7 μg/mL (4)), the effect of aspirin on collagen-induced platelet aggregation also decreased. The finding that a known anticoagulant inhibits collagen-induced platelet aggregation in a similar fashion to C3 and DF1 provides support for the use of C3 and DF1 as anticoagulants.

### Example 6

### Hemolytic Activity of NCL22

This example shows the hemolytic properties of NCL22.

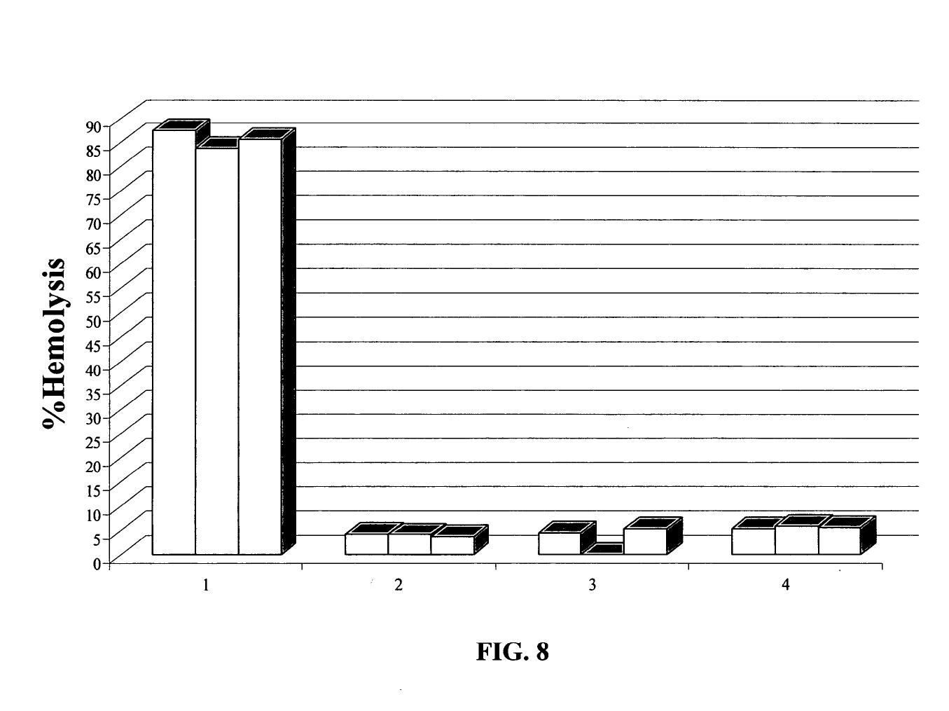

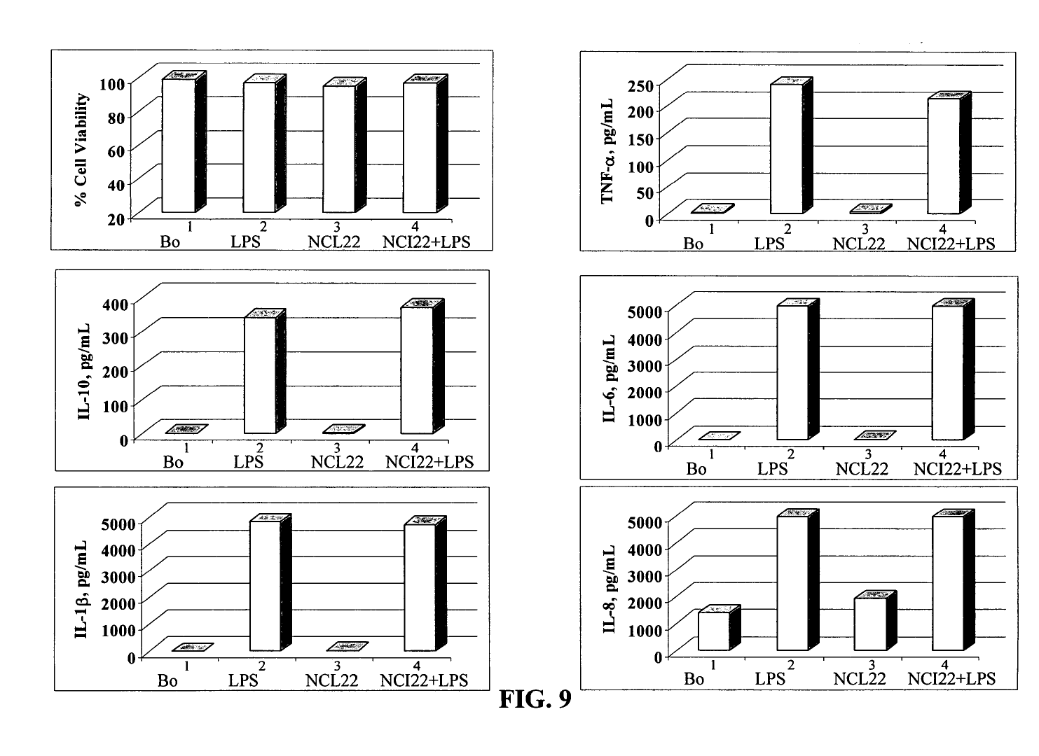

The effect of NCL22 on hemolysis was determined by use of the hemolysis assay described in detail in Example 1. The chemical structure of NCL22 is shown in FIG. 6. Triton-X (1) was used as a positive control. PBS (2) used to reconstitute the PAMAM dendrimer was used as a negative control. As illustrated in FIG. 8, neither a high concentration (1 mg/mL; 3) nor a low concentration (0.0156 mg/mL; 4) of NCL22 had any significant hemolytic activity. In contrast, Triton-X treatment resulted in greater than 80% hemolysis of the red blood cells. These studies suggest that NCL22 does not affect the integrity of red blood cells.

### Example 7

### Effect of NCL22 on Cytokine Secretion

This example shows the effect of NCL22 on cytokine secretion.

The effect of NCL22 on cytokine secretion by peripheral blood mononuclear cells (PBMC) was determined by use of a cytokine induction assay as described in detail at http://ncl.cancer.gov/working_assay-cascade.asp which is hereby incorporated by reference as of Oct. 9, 2006. Briefly, lymphocytes were isolated from pooled human blood anti-coagulated with Li-heparin using Ficoll-Paque Plus solution (Amersham Biosciences, catalogue No. 17-1440-02). The cells were incubated with or without lipopolysaccharide (LPS) in the presence or absence of nanoparticles (such as NCL22) for 24 hours. After incubation, cell culture supernatants were collected an analyzed by cytometry bead arrays for the presence of IL-1β, TNFα, IL-12, IL-10, IL-8 and IL-6. The assay allowed for the measurement of the nanoparticle's (NCL22) ability to induce cytokines or to suppress cytokines induced by LPS. Three independent samples were prepared and analyzed in duplicate. Shown is a mean response (% CV<25%).

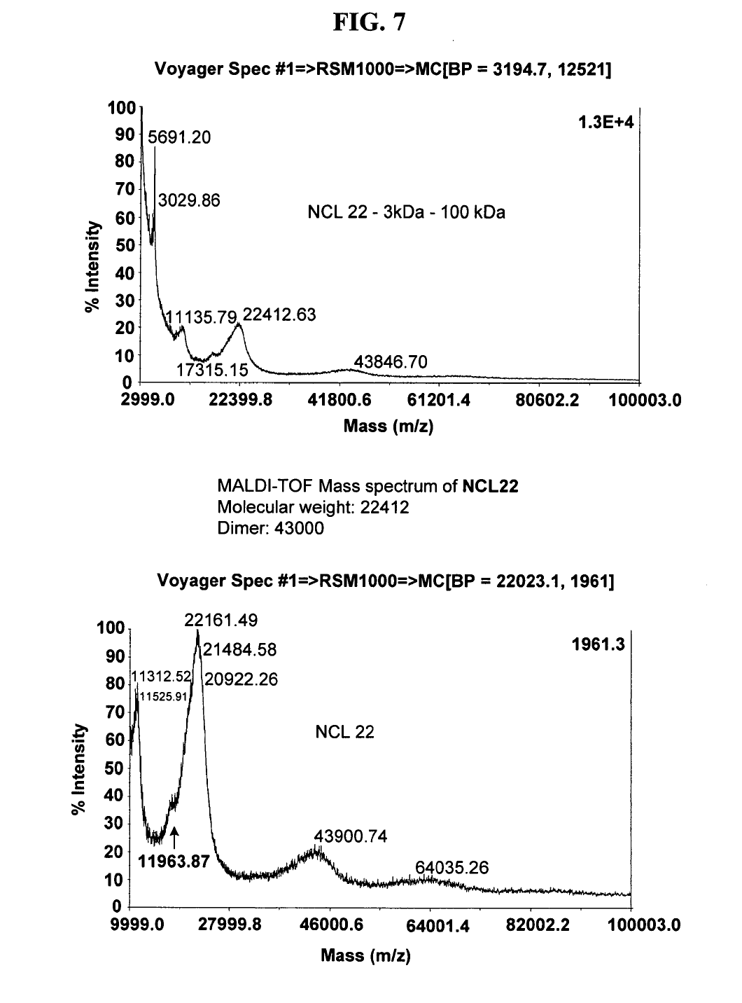

As illustrated in FIG. 7, NCL22 had no effect on cell viability in which the number of viable cells in the presence of NCL22 was approximately the equivalent to the number measured under control conditions. Further, NCL22 neither induced cytokine secretion nor did it interfere with LPS-induced cytokine secretion. Therefore, these studies suggest that NCL22 is not toxic to cytokines.

### Example 8

### Effect of NCL22 on Macrophage Activity

This example shows the effect of NCL22 on macrophage chemotactic and phagocytic activity.

Leukocyte recruitment is a central component of the inflammatory process, both in physiological host defense and in a range of prevalent disorders with an inflammatory component. In response to a complex network of proinflammatory signaling molecules (including cytokines, chemokines and prostaglandins), circulating leukocytes migrate from the bloodstream to the site of inflammation. The employed chemotactic assay represents an in vitro model, in which promyelocytic leukemia cells HL-60 were separated from control chemoattractant or test nanoparticles by a 3 μm filter; the cell migration through the filter was then monitored and number of migrated cells was quantitated using fluorescent dye calcein AM. The assay required 1.5 mL of a test nanomaterial (e.g., NCL22).

For the chemotactic studies, three independent samples were prepared and analyzed in duplicate. The assay required 1.5 mL of nanoparticles dissolved/re-suspended in starving medium (e.g., three 150 μL triplicates per sample). Heat-inactivated 20% FBS was used as a positive control (2) and PBS was used as a negative control (3). For the initial screen, the test concentration was selected based on results from general toxicity assays for the nanoparticle (see Example 15). HL-60 cells were placed into starving medium and incubated overnight at 37° C. in a 95% air, 5% CO₂. The following day, cells were counted using trypan blue and cell concentration was adjusted to 1×10⁶ viable cells per mL in the starving medium. Cell viability was equal to or greater than 90%. A 150 μL of positive control, negative control and test-nanomaterial (e.g., NCL22) were added to a feeding tray. Filter paper was inserted into a separate feeding tray. Fifty microliters of suspended cells were added per well of Multi-Screen filter plate (50,000 cells per well). An assay plate (plate including the Multi-Screen filter plate and feeding tray containing controls and test particles) was assembled. The plate was covered and allowed to incubate for 4 hours at 37° C. in a humidified incubator (5% CO₂, 95% air). During incubation, PBS was warmed to 37° C. and calcein AM was equilibrated to room temperature. After 4 hours of incubation, the Multi-Screen filter plate was removed and discarded. Fifty microliters of 1×PBS and 50 μL of calcein AM working solution were added to the appropriate wells, and 150 μL of 1×PBS plus 50 μL of calcein AM working solution were added to reagent background control wells on the feeding tray (the Calcein plate). Samples were allowed to incubate for 1 hour at 37° C. Solutions (180 μL) were transferred from the Calcein plate to corresponding wells on a Nunc optical bottom plate that was then read on a fluorescent plate reader at 485/535 nm. A Percent Coefficient of Variation was calculated for each control or test according to the following formula: % CV=SD/Mean×100%. Background chemotaxis=Mean FU_{SM/CAM wells}−Mean FU_{XM/PBS wells}−Mean FU_{reagent background control wells}. Sample chemotaxis=Mean FU_{TS/CAM wells}−Mean FU_{TS/PBS wells}−Mean FU_{reagent background control wells}. Comparison of sample chemotaxis to background chemotaxis was performed to evaluate chemotactic potential of test material (e.g., NCL22). In general, fold chemotaxis induction equal to or greater than 5 was considered positive. The aforementioned assay is described in detail at http://ncl.cancer.gov/working_assay-cascade.asp which is hereby incorporated by reference as of Oct. 9, 2006.

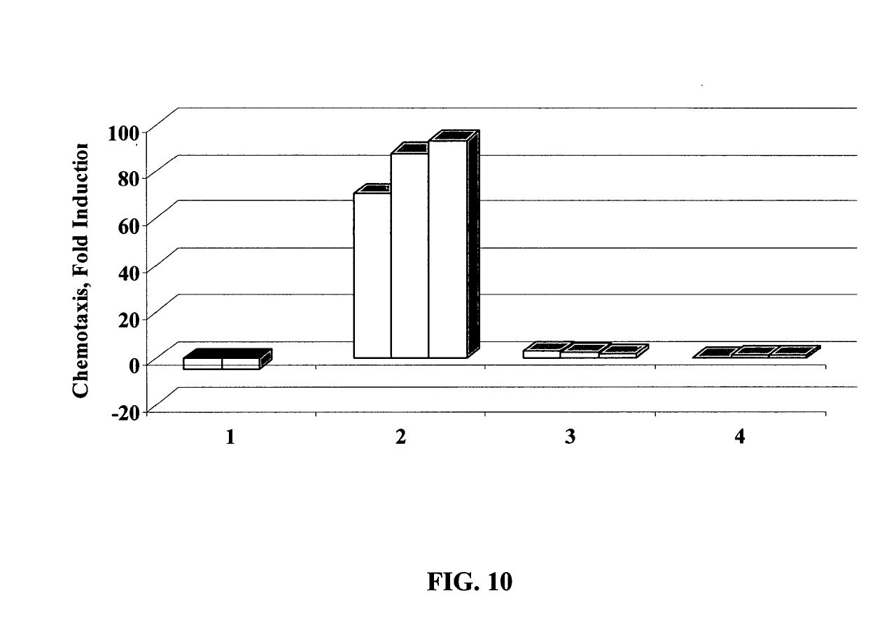

As illustrated in FIG. 10, macrophage chemotaxis was not induced by NCL22 at either a low concentration (4, 0.0156 mg/mL) or a high concentration (3, 1 mg/mL).

The ability of NCL22 to be internalized by macrophages via phagocytosis was evaluated by use of a phagocytosis assay. Briefly, the phagocytosis assay required 600 μL of nanoparticles dissolved/resuspended in PBS (e.g., three 100 μL duplicates per sample). Zymosan A (2 mg/mL) was used as a positive control (1) and PBS as a negative control. For the initial screen, 1 mg of nanoparticles dissolved in 0.5 mL of PBS was used. To test the ability of nanoparticles to interfere with phagocytosis of zymosan A, zymosan A was reconstituted in nanoparticles to have a final 2 mg/mL final concentration of zymosan. For either the positive control or zymosan A with nanoparticles studies, the samples were allowed to react with serum/plasma for 30 minutes at 37° C. Following incubation, the samples were rinsed with PBS, subject to centrifugation, and re-suspended in PBS to a final concentration of 2 mg/mL zymosan A.

HL-60 cells were utilized in the phagocytosis assay. HL-60 is a non-adherent promyelocytic cell line derived from a patient with acute promyelocytic leukemia (Collins, S. J. et al. Proc. Natl. Acad. Sci. U.S.A. 75 (5): 2458-62, 1978). Cell concentration was not allowed to exceed 1×10⁶ cells/mL. Prior to performing the assay, cells were counted by typan blue and cell viability was confirmed to be greater or equal to 90%. Cell concentration was adjusted to 1×10⁷ per mL by spinning the cell suspension down and reconstituting the cells in complete medium (medium containing 10% heat inactivated FBS, 2 mM L-glutamine, 50 μM β-mercaptoethanol, 100 U/mL penicillin 100 μg/mL streptomycin sulfate). Cells were maintained at room temperature. One hundred microliters of controls and test-nanoparticles (NCL22) were added to the appropriate wells in the 96 well test plate. Test samples were prepared in triplicate whereas the positive and negative controls were prepared in duplicate. One hundred microliters of a working luminol solution was added to each well containing sample. The plate was maintained at 37° C. during sample aliquoting. One hundred microliters of cell suspension was added per well on the 96 well white plate and the kinetic reading a luminescence plate reader was started immediately. A percent coefficient of variation was used to control precision and calculated for each control or test sample according to the following formula: % CV=SD/Mean×100%. Fold phagocytosis induction (FPI)=Mean RLUsample/Mean RLUnegative control. FPI of the positive control observed during assay quantification was greater than or equal to 400. The negative control was considered to be negative if RLU was less than or equal to 2000. The aforementioned assay is described in detail at http://ncl.cancer.gov/working_assay-cascade.asp which is hereby incorporated by reference as of Oct. 9, 2006.

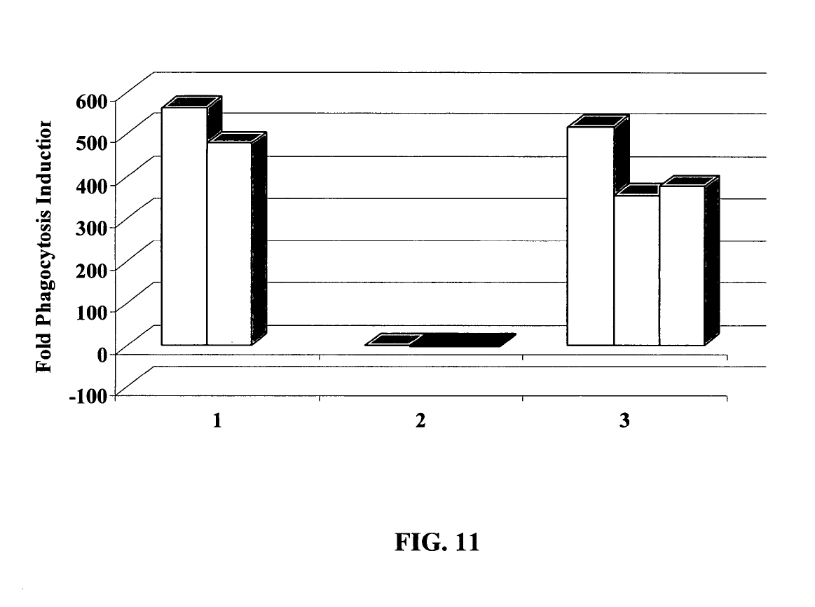

As illustrated in FIG. 11, NCL22 was not internalized by macrophages via phagocytosis (2). Further, NCL22 (1 mg/mL) did not affect phagocytic uptake of Zymosan-A (3, NCL22 1 mg/mL and Zymosan-A 2 mg/mL).

These studies suggest that NCL22 (up to 1 mg/mL) does not appear to be toxic to macrophages.

### Example 9

### Effect of NCL22 on Leukocyte Proliferation

This example shows the effect of NCL22 on leukocyte proliferation.

The effect of NCL22 at a low concentration (0.0156 mg/mL) and high concentration (1 mg/mL) was used to evaluate potential particles' toxicity to peripheral blood leukocytes. For the initial screening, the test concentration of the nanoparticle (NCL22) was selected based on results from general toxicity assays (see Example 15). A nanomaterial such as NCL22 that revealed toxicity in general toxicity assays was tested at two concentrations selected at the low and high end of the dose response curve. A nanomaterial that did not reveal toxicity in the general toxicity assays was tested at one concentration equal to the highest does tested in general toxicity assay. Human blood obtained from at least 3 donors was anti-coagulated with Li-heparin. Freshly drawn blood was placed into 15 or 50 mL conical centrifuge tube and an equal volume of room-temperature PBS was added and the contents were mixed well. The Ficoll-Paque Plus (Amersham Biosciences, catalogue no: 17-1440-02) was slowly layer underneath the blood/PBS mixture by placing the tip of the pipet containing the Ficoll-Paque solution at the bottom of the blood sample tube. Alternatively, the blood/PB mixture could be slowly layered over the solution. Three mL of Ficoll-Paque solution was used per 4 mL of blood/PBS mixture. The solution was then centrifuged for 30 minutes at 900 g, 18-20° C. (without a brake). Using a sterile pipet, the upper layer containing plasma and platelets was removed discarded. The mononuclear cell layer was then transferred into another centrifuge tube using a fresh sterile pipet. Cells were washed using an excess of HBSS and then, subjected to centrifugation for 10 min at 400 g, 18-20° C. The supernatant was then discarded and the wash step was repeated. Cells were then re-suspended in RPMI-1640 medium (Invitrogen, catalogue no: 11875-119). Cells were diluted to 1:5 or 1:10 with trypan blue. Cells were then counted and viability determined by using trypan blue exclusion. Cell concentration was adjusted to 1×10⁶ cells/mL using complete RPMI medium. One hundred microliters of control or test samples and then 100 μL of cell suspension were dispensed into each well of a 96 well round bottom plate. Plate was gently mixed and then allowed to incubate for 3 days in a humidified 37° C., 5% CO₂ incubator. After three days, the plate was centrifuged for 5 minutes at 700 g. Medium was then aspirated leaving cells and approximately 50 μL of medium behind. One hundred and fifty microliters of fresh medium and 50 μL of MTT (3-(4,5-dimethyl-2-thiazolyl)-2,5-diphenyl-2H-terazolium bromide; Sigma catalogue no: M5655) was then added to each well. The plate was then covered with aluminum foil and incubated in a humidified 37° C., 5% CO₂ incubator for 4 hours. At the appropriate time, the plate was removed from the incubator and spun at 700 g for 5 minutes. Media and MTT were aspirated and 200 μL of DMSO followed by 25 μL of glycine buffer was added to all wells. The plate was then read at 570 nm. A Percent Coefficient of Variation was calculated for each control or test according to the following formula: % CV=SD/Mead×100%. A % cell proliferation was calculated as (Mean ODₛₐₘₚₗₑ−MeanOD_{negative control})×100. Percent Proliferation of Inhibition was calculated as Mean OD_{positive Control}−Mean OD Positive_{Control+Nanoparticles}/Mean OD_{positive Control}×100%. This procedure is described in detail at http://ncl.cancer.gov/working_assay-cascade.asp which is hereby incorporated by reference as of Oct. 9, 2006.

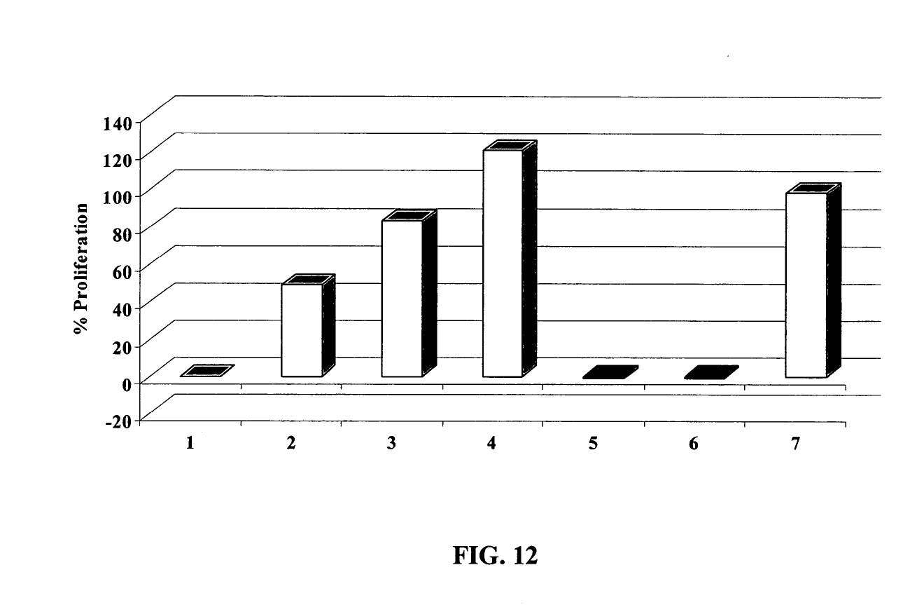

Three independent samples were prepared and analyzed in duplicate. Shown is a mean response (% CV<25%). Phytohemaglutinin-M (PHA-M) was used as positive control for proliferation induction and PBS was used as a negative control. As shown in FIG. 12, no cell proliferation was detected with PBS alone treatment (1). PHA-M, however, stimulated peripheral blood leukocyte proliferation in a dose-dependent manner (2, 5 μg/mL PHA-M; 3, 10 μg/mL PHA-M; and 4, 20 μg/mL). NCL22 had no effect on leukocyte proliferation (5, 1 mg/mL; 6, 0.0156 mg/mL). In addition, NCL22 did not suppress proliferation induced by PHA-M (7, NCL22 1 mg/Ml and PHA-M 10 μg/mL). These studies suggest that NCL22 is not toxic to peripheral blood leukocytes.

### Example 10

### Myelosuppressive Activity of NCL22

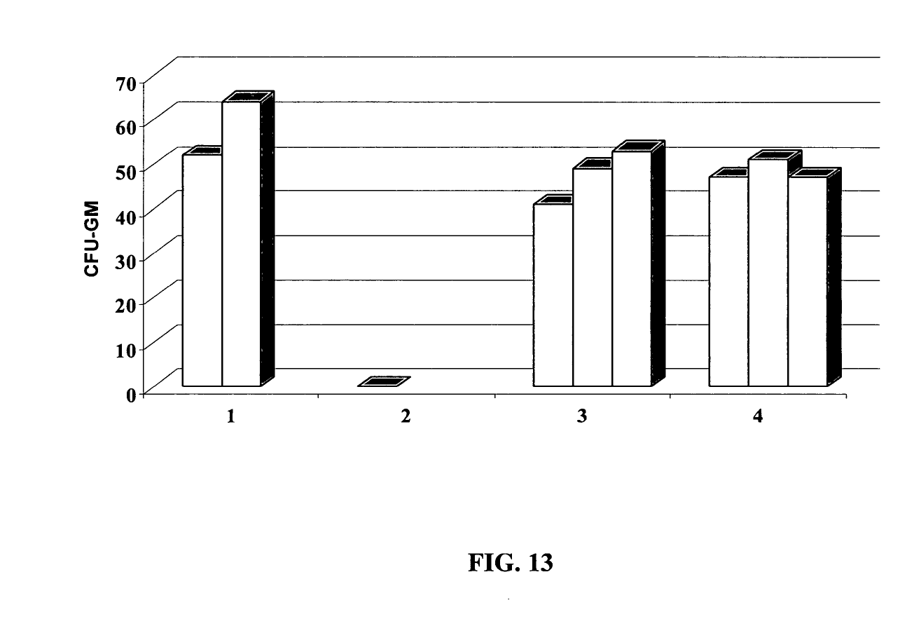

This example illustrates the lack of myelosuppressive activity of NCL22. The effect of NCL22 on myelosuppression was evaluated by use of the CFU-GM assay described in detail in Example 1. Briefly, three independent samples were prepared and analyzed in duplicate. As illustrated in FIG. 13, NCL22 did not have myelosuppressive activity at either a high (3, 1 mg/mL) or low (4, 0.0156 mg/mL) concentration as the effect of NCL22 at either concentration was similar to the effect observed in the negative control (1). In contrast, the positive control demonstrated significant myelosuppressive activity (2). These results demonstrate that NCL22 is not myelosuppressive.

### Example 11

### Effect of NCL22 on Platelet Aggregation

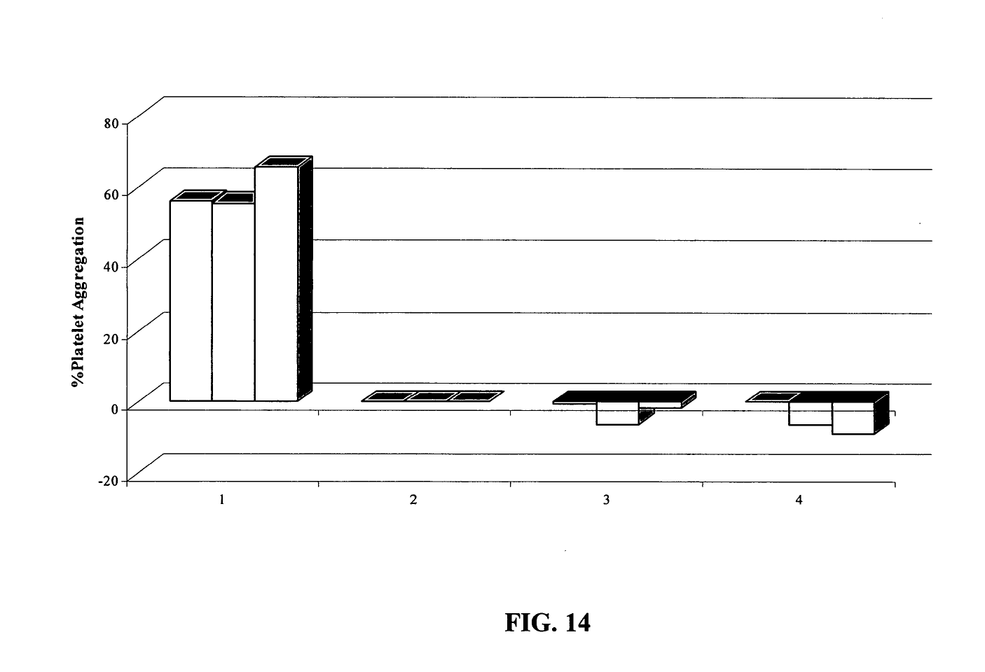

This example illustrates the effect of NCL22 on platelet aggregation. The platelet aggregation assay described in detail in Example 1 was used to evaluate the effects of NCL22 at either a high (3, 1 mg/mL) or low (4, 0.0156 mg/mL) concentration on platelet aggregation. Three independent samples were prepared for each NCL22 concentration and analyzed in duplicate. As shown in FIG. 14, NCL22 is not capable of inducing platelet aggregation (3 and 4) as compared to the positive control (1) that induced greater than a 50% platelet aggregation.

### Example 12

### Effect of NCL22 on Coagulation

This example illustrates the effect of NCL22 on coagulation.

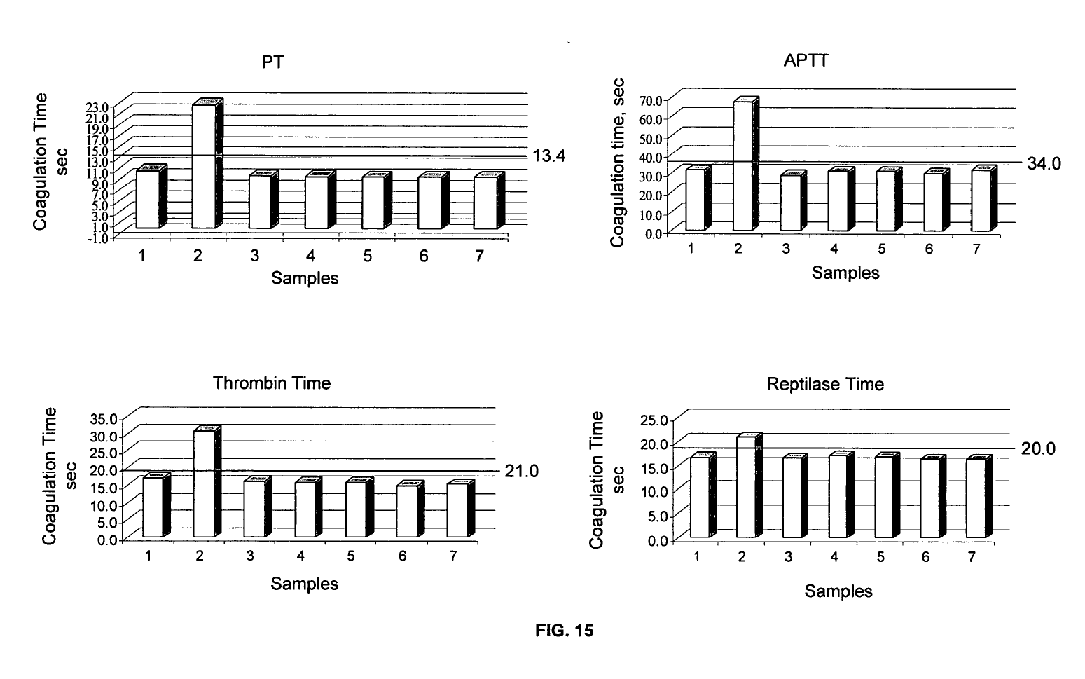

NCL22 at a high (1 mg/mL) concentration was used to evaluate NCL22 effects on various biochemical components of the blood coagulation cascade. Three independent samples were prepared and analyzed in duplicate. Normal plasma standard (1) and abnormal plasma standard (2) were used for the instrument control. Plasma was pooled from at least three donors was either untreated (3) or treated with NCL22 (4, 5, and 6). A Percent Coefficient of Variation was calculated for each control or test according to the following formula: % CV=SD/Mean×100%. Horizontal line indicates clinical standard cut-off for normal coagulation time for each of the tests. The aforementioned assay is described in detail at http://ncl.cancer.gov/working_assay-cascade.asp which is hereby incorporated by reference as of Oct. 9, 2006. The effect of NCL22 on prothrombin time (PT), activated partial thromboplastin time (APTT), thrombin time (TT) and reptilase time (RT) was evaluated. As illustrated in FIG. 15, NCL22 delayed coagulation time above clinically acceptable standard in APTT, thrombin time and reptilase time tests. These studies suggest that NCL22 is capable of modulating various biochemical components of the blood coagulation cascade.

### Example 13

### Effect of NCL22 on Protein Binding

This example illustrates the effect of the NCL22 on protein binding.

NCL22 was immobilized on CovaLink ELISA plate in order to achieve separation of particle-bound proteins from bulk plasma. Acetic acid was used as a negative control. During method development several blocking buffers were tested to block unspecific binding sites on ELISA plate. The detailed protocol used for analysis of NCL22 interaction with plasma proteins by two dimensional (2D) polyacrylamide gel electrophoresis (PAGE) is described at http://ncl.cancer.gov/working_assay-cascade.asp which is hereby incorporated by reference as of Oct. 9, 2006. In brief, NCL22 was incubated with pooled human plasma derived from healthy donors to allow for protein interaction and binding. Following a separation procedure, bound proteins were eluted from the nanoparticle surface and analyzed by 2D PAGE.

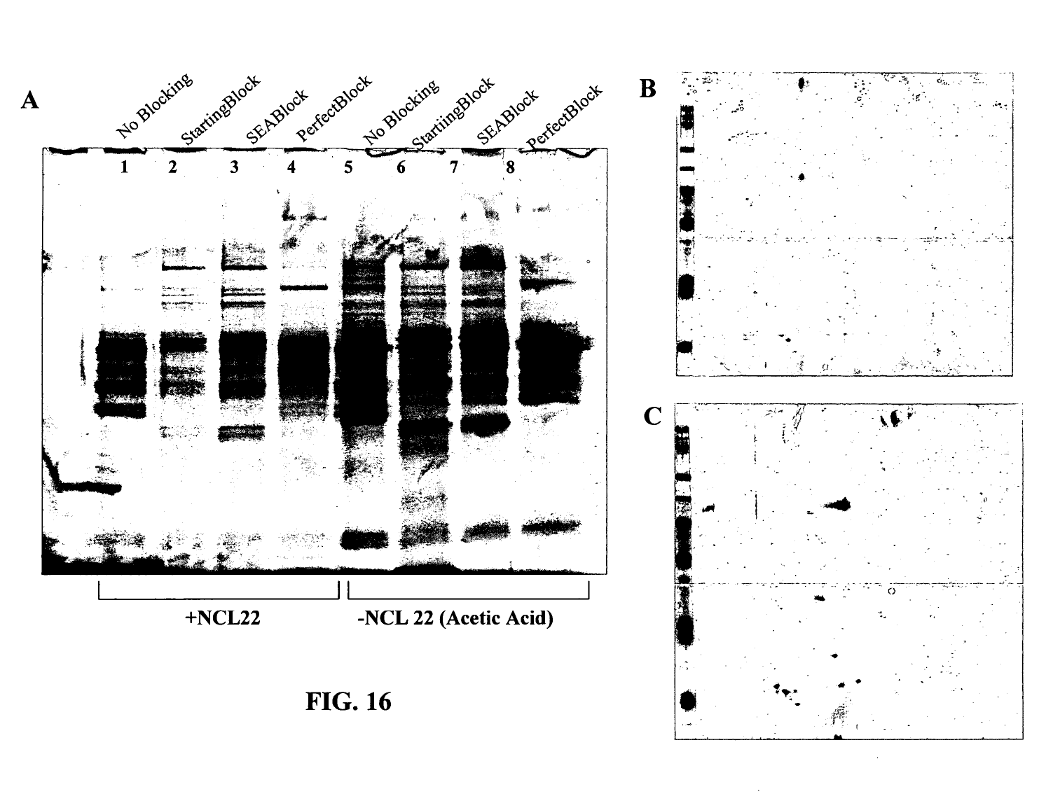

FIG. 16A illustrates the proteins isolated from plates coated with NCL22 following separation by polyacrylamide gel electrophoresis. FIG. 16B illustrates the proteins isolated from plates coated with acetic acid and FIG. 16C without any blocking buffers following separation by gel electrophoresis. These studies suggest that NCL22 can act as a protein binding blocker.

### Example 14

### Cytotoxicity of AF1, AF3, C3, DF1 and NCL22

This example illustrates the cytotoxic activities of AF1, AF3, C3, DF1 and NCL22.

The cytotoxicity of AF1, AF3, C3, DF1 and NCL22 on porcine proximal tubule epithelial cells (LLC-PK1) was determined by a method that includes a 3-(4,5-Dimethyl-2-thiazolyl)-2,5-diphenyl-2H-tetrazolium bromide (MTT) reduction and lactate dehydrogenase (LDH) release assays. MTT that is a yellow water-soluble tetrazolium dye that is reduced by live cells to a water-insoluble purple formazan. The amount of formazan can be determined by solubilizing the formazan in DMSO and measuring it spectrophotometrically. Comparisons between the spectra of treated and untreated cells can give a relative estimation of cytotoxicity. (See, Alley et al. Cancer Res. 48: 589-60, 1988 which is hereby incorporated by reference in its entirety). The LDH assay is based upon LDH (a cytoplasmic enzyme that is released into the cytoplasm upon cell lysis) oxidizing lactate to pyruvate, pyruvate reacting with a tetrazolium salt INT to form formazan, and the water-soluble formazan dye being detected spectrophotometrically. Thus, the LDH assay is a measure of membrane integrity. (See, Decker, T. & Lohmann-Matthes, M. L. J. Immunol. Methods 15: 61-69, 1988; Korzeniewski, C. & Callewaert, D. M. J. Immunol. Methods 64: 313-320, 1983, which are each incorporated by reference in their entirety.) The aforementioned assays are described in detail at http://ncl.cancer.gov/working_assay-cascade.asp which is hereby incorporated by reference as of Oct. 9, 2006.

Briefly, for assays employing porcine kidney epithelial cells, such cells were plated at 2.5×10-5 cells/mL in a 96 well plate. For assays employing HepG2 cells, cells were plated at 5.0×10-5 cells/mL in a 96 well plate. Cells were pre-incubated for 24 hours and then treated for 6, 24, or 48 hours with the test substance ranging in concentration from 1.0 to 0.004 mg/mL for porcine kidney epithelial cells and 5.0 to 0.02 mg/mL for HepG2 cells. Cytotoxicity was then determined by MTT and LDH assays.

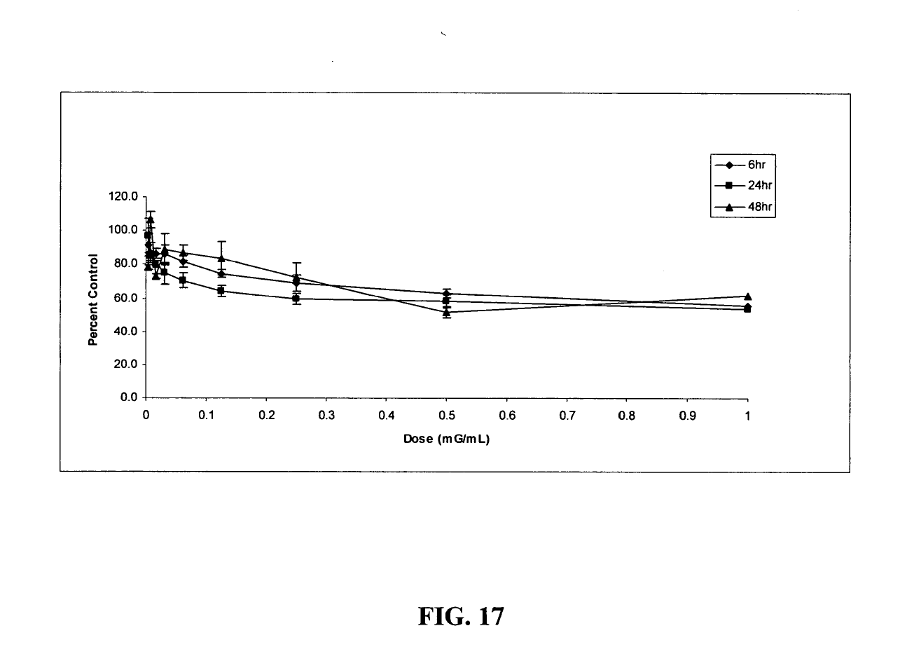

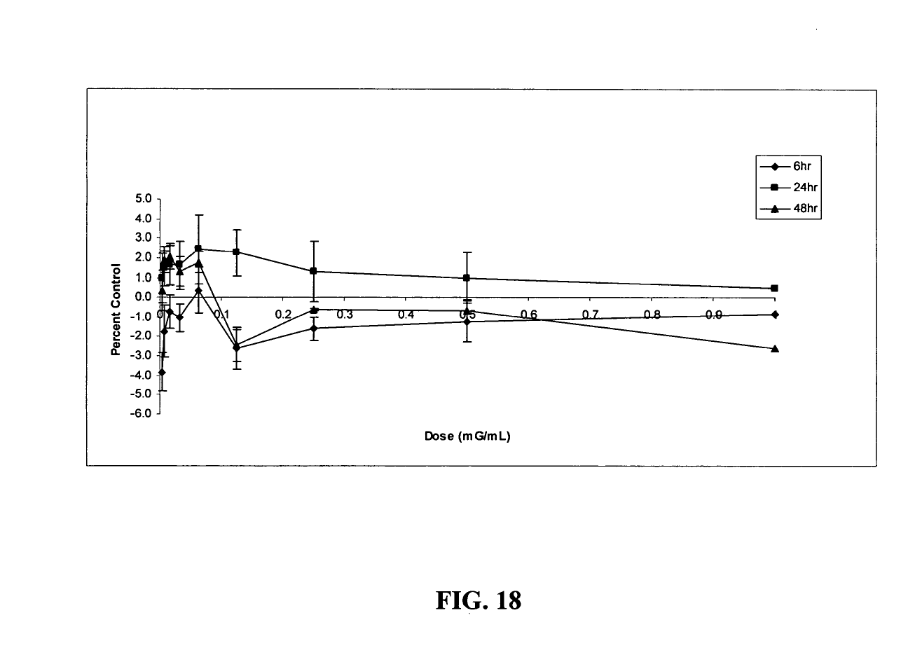

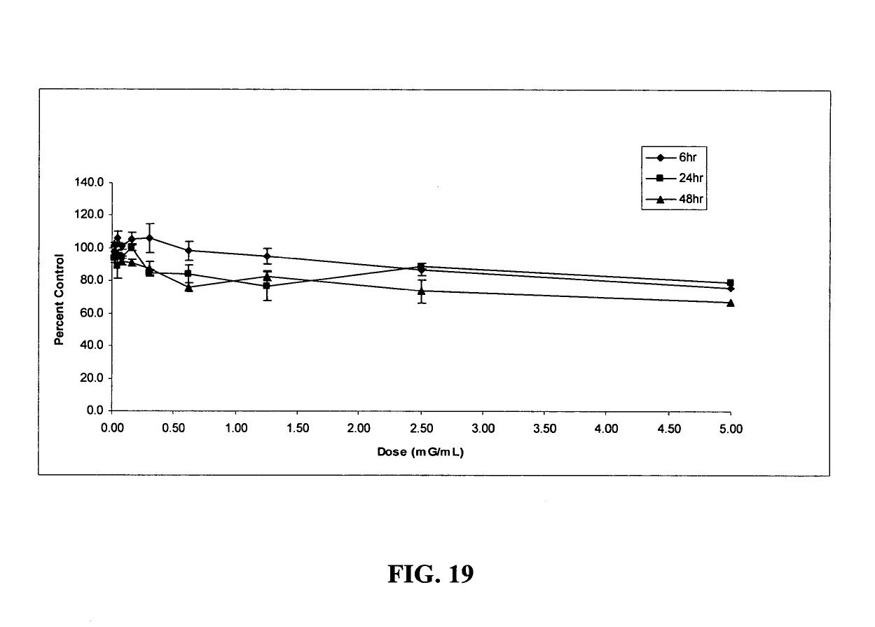

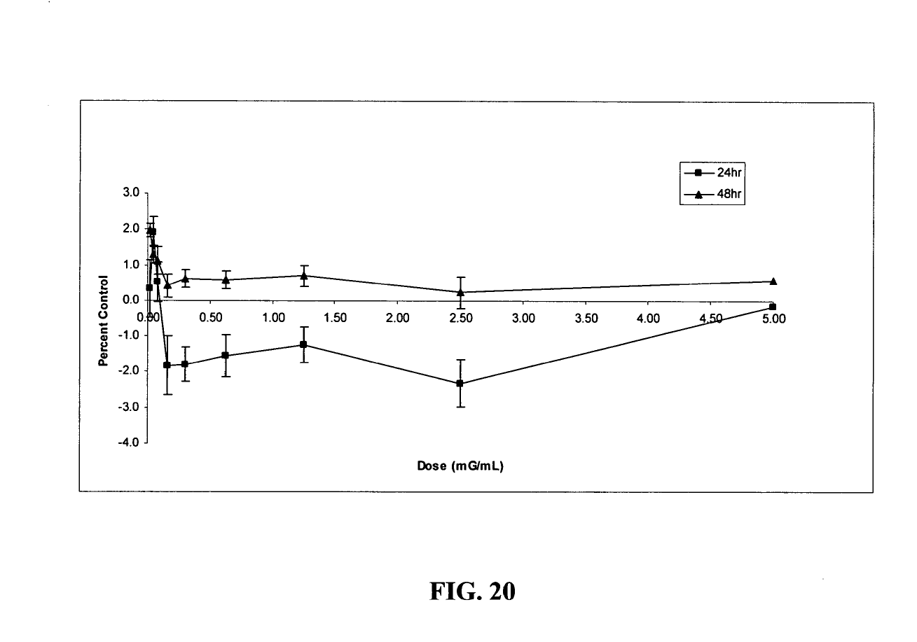

Table 1 below illustrates the LC₅₀ (the amount of a material, given all at once, which causes the death of 50% (one half) of porcine kidney epithelial cells) determined for C3, DF1, AF1, and AF3 with the MTT and LDH assays. FIG. 17 illustrates the cytotoxic effects of NCL22 on LDK-PK1 kidney epithelial cells according to a MTT cytotoxicity assay whereas FIG. 18 shows the cytotoxic effects of NCL22 on LDK-PK1 kidney epithelial cells according to a LDH cytotoxicity assay. In addition, the cytotoxic effects of NCL22 on HepG2 hepatic carcinoma cells according to a MTT cytotoxicity assay is shown in FIG. 19 and a LDH cytotoxicity assay in FIG. 20. These studies demonstrate that DF1 and AF3 are nontoxic to kidney cells. In contrast, C3, AF1 and NCL22 are minimally toxic to such cells. Further, NCL22 is also minimally toxic to liver cells.

| Sample | LC₅₀, mg/mL |
|---|---|
| C3 | 0.207 mg/mL |
| DF1 | Nontoxic |
| AF1 | 0.213 mg/mL |
| AF3 | nontoxic |

### Example 15

### Treatment of Blood Clotting Disorders with Nanoparticle-Based Anticoagulants

Based upon the teaching disclosed herein, a blood clotting disorder such as undesired platelet aggregation, thrombosis, or peripheral arterial occlusion may be treated by administering a therapeutic effective dose of a nanoparticle-based anticoagulant such as C3, a C3 analog, DF1, a DF1 analog, NCL22 or a NCL22 analog or combination thereof. In an example, a subject who has been diagnosed with a blood clotting disorder or has the potential to acquire a blood clotting disorder (e.g., a subject that is to have a medical device implanted) may be identified. Following subject selection, a therapeutic effective dose of the nanoparticle-based anticoagulant is administered to the subject. For example, a therapeutic effective dose of a substituted fullerene (such as C3, a C3 analog, DF1 or a DF1 analog) may be administered to the subject. In a further example, a therapeutic effective dose of NCL22 or a NCL analog may be administered to the subject. The nanoparticle-based anticoagulants are prepared and purified as described in Section III.B. The amount of the nanoparticle-based anticoagulant administered to prevent, reduce, inhibit, and/or treat the blood clotting disorder depends on the subject being treated, the severity of the disorder, and the manner of administration of the therapeutic composition. Ideally, a therapeutically effective amount of an agent is the amount sufficient to prevent, reduce, and/or inhibit, and/or treat the disorder in a subject without causing a substantial cytotoxic effect in the subject.

### Example 16

### Associating a Nanoparticle-Based Anticoagulant with a Medical Device

According to the teachings herein, one or more nanoparticle-based anticoagulants may be associated either by coating or impregnating a medical device such as a stent or catheter to prevent, reduce, inhibit, and/or treat a blood clotting disorder often associated with implantation of the medical device. The nanoparticle-based anticoagulant can include at least one of an anticoagulant substituted fullerene (such as C3, a C3 analog, DF1, a DF1 analog, or a mixture thereof), a PAMAM dendrimer (such as NCL22, a NCL22 analog) or a combination thereof. The nanoparticle-based anticoagulants are prepared and purified as described in Section III.B. In an example, the medical device may be partially or completely coated with the nanoparticle-based anticoagulant. For instance, the medical device can be partially coated with the nanoparticle-based anticoagulant such as at the points at which the medical device interacts with the subject's vessel, organ or tissue. Such configuration is believed to reduce undesired platelet formation often associated with implantation of the medical device while minimizing the amount of coating material and time required to prepare the device. In a further example, the medical device may be substantially coated with the nanoparticle-based anticoagulant. The nanoparticle-based anticoagulant is attached to the medical device as described in Section V. For example, any chemical or mechanical bond or force, including linking agents can be used to coat the device. Alternatively, the coating can be directly linked (tethered) to the first surface through silane groups.

In a further example, the medical device such as a stent or catheter is impregnated with at least one nanoparticle-based anticoagulant by the methods described in Section V. In one example, the medical device is at least partially impregnated with the anticoagulant so that at least one surface including the nanoparticle-based anticoagulant to enhance the interaction of the device with the organ, tissue, vessel, and the like in which the device is used. In another example, the nanostructured components (e.g., nanoparticle-based anticoagulant) are substantially impregnated throughout the device so that the multiple surfaces (such as the outer and inner surfaces) of the medical device include the nanoparticle-based anticoagulants.

In an additional example, the medical device is coated or impregnated with materials in addition to the disclosed nanoparticle-based anticoagulants to further enhance their bio-utility. Examples of suitable coatings are medicated coatings, drug-eluting coatings, hydrophilic coatings, and smoothing coatings as described in Section V.

An effective amount of the nanoparticle-based anticoagulant to be used in coating or impregnation will depend, at least, on the particular method of use, the subject being treated, the severity of the disorder, and the manner of administration of the therapeutic composition. For example, this can be the amount of a nanoparticle-based anticoagulant necessary to prevent, inhibit, reduce or relieve the blood clotting disorder such as thrombosis and/or one or more symptoms of the disorder such as undesired platelet aggregation associated with implantation of a medical device into a subject. Ideally, a therapeutically effective amount of the nanoparticle-based anticoagulant is an amount sufficient to prevent, inhibit, reduce or relieve the blood clotting disorder and/or one or more symptoms such as catheter obstruction caused by the disorder without causing a substantial cytotoxic effect on host cells.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for preventing or treating a blood clotting disorder, comprising:
administering a therapeutic effective amount of at least one nanoparticle-based anticoagulant to a subject afflicted with blood clotting disorder or potentially afflicted with a blood clotting disorder, wherein the at least one nanoparticle-based anticoagulant is a substituted fullerene, polyamidoamine (PAMAM) dendrimer or a combination or mixture thereof.

2. The method of claim 1, wherein the at least one nanoparticle-based anticoagulant is C3, a C3 analog, DF1, a DF1 analog, NCL22, a NCL22 analog, a substituted fullerene with a plurality of carboxy-terminated dendritic branches, a PAMAM dendrimer that has carboxy-terminated dendritic branches or a mixture thereof.

3. (canceled)

4. The method of claim 1, wherein the at least one nanoparticle-based anticoagulant is associated with at least one carrier or adjuvant.

5. The method of claim 1, wherein the at least one nanoparticle-based anticoagulant is administered in conjunction with insertion of an in-dwelling device into the subject, the in-dwelling device being at least one of a stent, a stent graft, a synthetic vascular graft, a heart valve, a catheter, a vascular prosthetic filter, a pacemaker, a pacemaker lead, a defibrilator, a patent foramen ovale (PFO) septal closure device, a vascular clip, a vascular aneurysm occluder, a hemodialysis graft, a hemodialysis catheter, an atrioventricular shunt, an aortic aneurysm graft device or components, a venous valve, a suture, a vascular anastomosis clip, an indwelling venous or arterial catheter, a vascular sheath or a drug delivery port.

6. The method of claim 5, wherein the at least one nanoparticle-based anticoagulant is administered locally at an implantation site of the in-dwelling device.

7. (canceled)

8. The method of claim 1, wherein administration is via an in-dwelling device.

9. The method of claim 8, wherein the in-dwelling device is at least partially coated with the nanoparticle-based anticoagulant by the use of at least one of a linking agent, chemical reactive group, or combination thereof.

10. The method of claim 9, wherein the at least one linking agent, chemical reactive group, or combination thereof is selected from the group consisting of a substituted silane, diacetylene, acrylate, acrylamide, vinyl, styryl, silicon oxide, boron oxide, phosphorus oxide, N-(3-aminopropyl)-3-mercapto-benzamide, 3-aminopropyl-trimethoxysilane, 3-mercaptopropyl-trimethoxysilane, 3-maleimidopropyl-trimethoxysilane, 3-hydrazidopropyl-trimethoxysilane, hydroxysuccinimides, maleimides, haloacetyls, pyridyl disulfides, hydrazines, and ethyldiethylamino propylcarbodiimide.

11. The method of claim 8, wherein the in-dwelling device is at least partially coated with the nanoparticle-based anticoagulant by directly linking the nanoparticle-based anticoagulant to the in-dwelling device through silane groups.

12. The method of claim 8, wherein the in-dwelling device is at least partially impregnated with the nanoparticle-based anticoagulant so that at least one surface of the device that includes the anticoagulant interacts with the organ, tissue or vessel in which the device is being implanted.

13. The method of claim 8, wherein the in-dwelling device is at least partially coated or impregnated with the nanoparticle-based anticoagulant and an additional reagent that enhances the bio-utility of the device, wherein the additional reagent is at least one of a medicated coating, drug-eluting coating, hydrophilic coating, smoothing coating or a combination thereof.

14. The method of claim 1, wherein the at least one nanoparticle-based anticoagulant is administered intravenously to the subject.

15. The method of claim 1, wherein the at least one nanoparticle-based anticoagulant is administered to a subject afflicted with thrombosis, to a subject potentially afflicted with thrombosis, to a subject afflicted with peripheral arterial occlusion, to a subject potentially afflicted with peripheral arterial occlusion, to prevent or treat a blood clotting disorder associated with implantation of an in-dwelling device into the subject, to prevent or treat undesired platelet aggregation associated with implantation of an in-dwelling device into the subject, to prevent or treat catheter obstruction or any combination thereof.

16.-20. (canceled)

21. The method of claim 1, wherein the blood clotting disorder is undesired platelet aggregation.

22.-38. (canceled)

39. A pharmaceutical composition for inhibiting undesired platelet aggregation, comprising:
an amount of at least one nanoparticle-based anticoagulant therapeutically effective for inhibiting undesired platelet aggregation; and
at least one carrier or adjuvant,
wherein the at least one nanoparticle-based anticoagulant is a substituted fullerene, polyamidoamine (PAMAM) dendrimer or a combination or mixture thereof.

40. The pharmaceutical composition of claim 39, wherein the at least one nanoparticle-based anticoagulant is C3, a C3 analog, DF1, a DF1 analog, NCL22, a NCL22 analog, or a mixture thereof.

41. (canceled)

42. The pharmaceutical composition of claim 39, wherein the pharmaceutical composition is for inhibiting platelet aggregation induced by collagen.

43. A device configured for implantation or insertion into a subject, the device, comprising:
at least one structural element comprising an amount of at least one nanoparticle-based anticoagulant therapeutically effective for inhibiting platelet aggregation,
wherein the at least one nanoparticle-based anticoagulant is a substituted fullerene, polyamidoamine (PAMAM) dendrimer or a combination or mixture thereof.

44. The device of claim 43, wherein the at least one substituted fullerene nanoparticle-based anticoagulant is C3, a C3 analog, DF1, a DF1 analog, NCL22, a NCL22 analog, or a mixture thereof.

45. (canceled)

46. The device of claim 43, wherein the device is an in-dwelling device, the in-dwelling device is at least one of a stent, a stent graft, a synthetic vascular graft, a heart valve, a catheter, a vascular prosthetic filter, a pacemaker, a pacemaker lead, a defibrilator, a patent foramen ovale (PFO) septal closure device, a vascular clip, a vascular aneurysm occluder, a hemodialysis graft, a hemodialysis catheter, an atrioventricular shunt, an aortic aneurysm graft device or components, a venous valve, a suture, a vascular anastomosis clip, an indwelling venous or arterial catheter, a vascular sheath or a drug delivery port.

47. The device of claim 46, wherein the in-dwelling device is at least partially coated with the nanoparticle-based anticoagulant by use of at least one of a linking agent, chemical reactive group, or combination thereof.

48. The device of claim 47, wherein the at least one linking agent, chemical reactive group, or combination thereof is selected from the group consisting of a substituted silane, diacetylene, acrylate, acrylamide, vinyl, styryl, silicon oxide, boron oxide, phosphorus oxide, N-(3-aminopropyl)-3-mercapto-benzamide, 3-aminopropyl-trimethoxysilane, 3-mercaptopropyl-trimethoxysilane, 3-maleimidopropyl-trimethoxysilane, 3-hydrazidopropyl-trimethoxysilane, hydroxysuccinimides, maleimides, haloacetyls, pyridyl disulfides, hydrazines, and ethyldiethylamino propylcarbodiimide.

49. The device of claim 46, wherein the in-dwelling device is at least partially coated with the nanoparticle-based anticoagulant by directly linking the anticoagulant to the in-dwelling device through silane groups.

50. The device of claim 46, wherein the in-dwelling device is at least partially impregnated with the nanoparticle-based anticoagulant so that at least one surface of the device that includes the anticoagulant interacts with the organ, tissue or vessel in which the device is being implanted.

51. The device of claim 46, wherein the in-dwelling device is at least partially coated or impregnated with the at least one nanoparticle-based anticoagulant and an additional reagent that enhances the bio-utility of the device, wherein the additional reagent is at least one of a medicated coating, drug-eluting coating, hydrophilic coating, smoothing coating or a combination thereof.

52.-58. (canceled)
